SEQUENCE LISTING (72 sequences)

SEQ ID NO: 1 - DNA, 2254 bp, Homo sapiens
agagtgcacccgaatccacgggctcggaggcagcagccatctctcggccatagggcaggccagctggcgccgggggctattttgggcggcgggcaatgatggtgaccgcaaggcgaccttgtaaggcatttcccccctgactcccttccccgagcctctgcccgggggtcctagcgccgctttctcagccatcccgcctacaacttagccgtccacaacaggatcatctgatcgcgtgcgcccgggctacgatctgcgaggcccgcggaccttgacccggcattgaccgccaccgccccccaggtccgtagggaccaaagaaggggcgggaggaagactgtcacgtggcgccggagttcacgtgactcgtacacatgacttccagtccccgggcgcctcctggagagcaaggacgcgggggagcagagatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggccgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttcaaaaaaaaaaaaaaaa

SEQ ID NO: 2 - protein, 498 aa, Homo sapiens
MTSSPRAPPGEQGRGGAEMIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 3 - DNA, 2088 bp, Homo sapiens
gagctacttgaagaccaattagagtccgggaagcgcggcggggcctccagaccggggcgggcttaagggtgacatctgcgctttaaagggtccgggtcagctgactcccgactctgtggagtctagctgccagggtcgcggcagctgcggggagagatgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctgagctgtgccactgccacaggggtattctgccttcaggactctgccttcaggaacacgggtctgtagagggtctgctggagacgcctgaaagacagttccatcttcctttagactccagccttggcaaaatcaccttccctttaccagggaaatcacttcctttaggactgaaagctaggcgtcctctcccacaaaaaagtcctgccctcatctgagaatactgtctttccatatggctaagtgtggccccaccaccctctctgccctcccgggacattgattggtcctgtcttgggcaggtctagtgagctgtagaattgaatcaatgtgaactcagggaactggggaaggctgagcctcctctttggtgttgcggtaagataaccgacagggctggtgaaagtccccagatggcaggatatttggtttcagagtaaggactaggtgcaccaccatgactgactatcaatcaaaatgtttgtaacttaaaatttttaatgaaggataatgaatatttgtagagtctctatggttctgtcaatgcacatcttcgtgtctgttttcctcatgtatccttgtgagcctgggtgagttctggggagagacctgatgtgcgtactgcctgtgaaaatctgactttggcaaatcaaatcctcttttccttttgaaaaaaaaaaaaaaaaaa

SEQ ID NO: 4 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 5 - DNA, 3540 bp, Homo sapiens
ggtggtggaatatagagctcatgtgatccgtcacatgacagcagatccgcggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacgtagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaaaccaaaaaaa

SEQ ID NO: 6 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 7 - DNA, 3783 bp, Homo sapiens
ggggcggggccgggagggtacttagggccggggctggcccaggctacggcggctgcagggctccggcaaccgctccggcaacgccaaccgctccgctgcgcgcaggctgggctgcaggctctcggctgcagcgctgggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 8 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 9 - DNA, 1825 bp, Homo sapiens
acacatgctgcatacacacagaaacactgcaaatccactgcctccttccctcctccctacccttccttctctcagcatttctatccccgcctcctcctcttacccaaattttccagccgatcactggagctgacttccgcaatcccgatggaataaatctagcacccctgatggtgtgcccacactttgctgccgaaacgaagccagacaacagatttccatcagcaggatgtgggggctcaaggttctgctgctacctgtggtgagctttgctctgtaccctgaggagatactggacacccactgggagctatggaagaagacccacaggaagcaatataacaacaaggtggatgaaatctctcggcgtttaatttgggaaaaaaacctgaagtatatttccatccataaccttgaggcttctcttggtgtccatacatatgaactggctatgaaccacctgggggacatgaccagtgaagaggtggttcagaagatgactggactcaaagtacccctgtctcattcccgcagtaatgacaccctttatatcccagaatgggaaggtagagccccagactctgtcgactatcgaaagaaaggatatgttactcctgtcaaaaatcagggtcagtgtggttcctgttgggcttttagctctgtgggtgccctggagggccaactcaagaagaaaactggcaaactcttaaatctgagtccccagaacctagtggattgtgtgtctgagaatgatggctgtggagggggctacatgaccaatgccttccaatatgtgcagaagaaccggggtattgactctgaagatgcctacccatatgtgggacaggaagagagttgtatgtacaacccaacaggcaaggcagctaaatgcagagggtacagagagatccccgaggggaatgagaaagccctgaagagggcagtggcccgagtgggacctgtctctgtggccattgatgcaagcctgacctccttccagttttacagcaaaggtgtgtattatgatgaaagctgcaatagcgataatctgaaccatgcggttttggcagtgggatatggaatccagaagggaaacaagcactggataattaaaaacagctggggagaaaactggggaaacaaaggatatatcctcatggctcgaaataagaacaacgcctgtggcattgccaacctggccagcttccccaagatgtgactccagccagccaaatccatcctgctcttccatttcttccacgatggtgcagtgtaacgatgcactttggaagggagttggtgtgctatttttgaagcagatgtggtgatactgagattgtctgttcagtttccccatttgtttgtgcttcaaatgatccttcctactttgcttctctccacccatgacctttttcactgtggccatcaggactttccctgacagctgtgtactcttaggctaagagatgtgactacagcctgcccctgactgtgttgtcccagggctgatgctgtacaggtacaggctggagattttcacataggttagattctcattcacgggactagttagctttaagcaccctagaggactagggtaatctgacttctcacttcctaagttcccttctatatcctcaaggtagaaatgtctatgttttctactccaattcataaatctattcataagtctttggtacaagtttacatgataaaaagaaatgtgatttgtcttcccttctttgcacttttgaaataaagtatttatctcctgtctacagtttaataaatagcatctagtacacattcaaaaaaaaaaaaaaaa

SEQ ID NO: 10 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 11 - DNA, 1730 bp, Homo sapiens
ggcggtgccggccgaacccagacccgaggttttagaagcagagtcaggcgaagctgggccagaaccgcgacctccgcaaccttgagcggcatccgtggagtgcgcctgcgcagctacgaccgcagcaggaaagcgccgccggccaggcccagctgtggccggacagggactggaagagaggacgcggtcgagtaggtgtgcaccagccctggcaacgagagcgtctaccccgaactctgctggccttgaggtggggaagccggggagggcagttgaggaccccgcggaggcgcgtgactggttgagcgggcaggccagcctccgagccgggtggacacaggttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgtagtggtggggcttctttctatttttgatgcactgaatttttgtgtaataaagaacataattgggctctaagccataaaaaaaaaaaaaaaaaa

SEQ ID NO: 12 - protein, 333 aa, Homo sapiens [sequence omitted]

SEQ ID NO: 13 - protein, 5 aa, Artificial Sequence
  signal peptide
  misc_feature (2)..(3): Xaa can be any naturally occurring amino acid
DXXLL

SEQ ID NO: 14 - protein, 8 aa, Artificial Sequence
  signal peptide
  misc_feature (3)..(5): Xaa can be any naturally occurring amino acid
DEXXXLLI

SEQ ID NO: 15 - protein, 4 aa, Artificial Sequence
  signal peptide
  misc_feature (2)..(3): Xaa can be any naturally occurring amino acid
  MISC_FEATURE (4)..(4): Xaa may be an amino acid with a bulky hydrophobic side chain, such as Ile, Phe, Leu, Val, and Met
YXXX

SEQ ID NO: 16 - protein, 11 aa, Artificial Sequence
  signal peptide
SFHDDSDEDLL

SEQ ID NO: 17 - protein, 11 aa, Artificial Sequence
  signal peptide
EESEERDDHLL

SEQ ID NO: 18 - protein, 11 aa, Artificial Sequence
  signal peptide
GYHDDSDEDLL

SEQ ID NO: 19 - protein, 11 aa, Artificial Sequence
  signal peptide
ITGFSDDVPMV

SEQ ID NO: 20 - protein, 11 aa, Artificial Sequence
  signal peptide
ASVSLLDDELM

SEQ ID NO: 21 - protein, 11 aa, Artificial Sequence
  signal peptide
ASSGLDDLDLL

SEQ ID NO: 22 - protein, 11 aa, Artificial Sequence
  signal peptide
VQNPSADRNLL

SEQ ID NO: 23 - protein, 11 aa, Artificial Sequence
  signal peptide
NALSWLDEELL

SEQ ID NO: 24 - protein, 7 aa, Artificial Sequence
  signal peptide
DERAPLI

SEQ ID NO: 25 - protein, 7 aa, Artificial Sequence
  signal peptide
TERERLL

SEQ ID NO: 26 - protein, 7 aa, Artificial Sequence
  signal peptide
SETERLL

SEQ ID NO: 27 - protein, 7 aa, Artificial Sequence
  signal peptide
TDRTPLL

SEQ ID NO: 28 - protein, 7 aa, Artificial Sequence
  signal peptide
EETQPLL

SEQ ID NO: 29 - protein, 7 aa, Artificial Sequence
  signal peptide
DDQRDLI

SEQ ID NO: 30 - protein, 7 aa, Artificial Sequence
  signal peptide
NEQLPML

SEQ ID NO: 31 - protein, 5 aa, Artificial Sequence
  signal peptide
GYQTI

SEQ ID NO: 32 - protein, 5 aa, Artificial Sequence
  signal peptide
GYEQF

SEQ ID NO: 33 - protein, 5 aa, Artificial Sequence
  signal peptide
GYQTL

SEQ ID NO: 34 - protein, 5 aa, Artificial Sequence
  signal peptide
GYQSV

SEQ ID NO: 35 - protein, 5 aa, Artificial Sequence
  signal peptide
GYEVM

SEQ ID NO: 36 - protein, 5 aa, Artificial Sequence
  signal peptide
AYQAL

SEQ ID NO: 37 - protein, 5 aa, Artificial Sequence
  signal peptide
NYHTL

SEQ ID NO: 38 - protein, 5 aa, Artificial Sequence
  signal peptide
GYQRI

SEQ ID NO: 39 - protein, 5 aa, Artificial Sequence
  signal peptide
GYDQL

SEQ ID NO: 40 - protein, 5 aa, Artificial Sequence
  signal peptide
GYKEI

SEQ ID NO: 41 - protein, 5 aa, Artificial Sequence
  signal peptide
GYRHV

SEQ ID NO: 42 - DNA, 2300 bp, Homo sapiens
agagtgcacccgaatccacgggctcggaggcagcagccatctctcggccatagggcaggccagctggcgccgggggctattttgggcggcgggcaatgatggtgaccgcaaggcgaccttgtaaggcatttcccccctgactcccttccccgagcctctgcccgggggtcctagcgccgctttctcagccatcccgcctacaacttagccgtccacaacaggatcatctgatcgcgtgcgcccgggctacgatctgcgaggcccgcggaccttgacccggcattgaccgccaccgccccccaggtccgtagggaccaaagaaggggcgggaggaagactgtcacgtggcgccggagttcacgtgactcgtacacatgacttccagtccccgggcgcctcctggagagcaaggacgcgggggagcagaggtgagctggcaccggaggctggaggggatccccgagcccgggatcgatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggccgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttcaaaaaaaaaaaaaaaa

SEQ ID NO: 43 - protein, 480 aa, Homo sapiens
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 44 - DNA, 2208 bp, Homo sapiens
agagtgcacccgaatccacgggctcggaggcagcagccatctctcggccatagggcaggccagctggcgccgggggctattttgggcggcgggcaatgatggtgaccgcaaggcgaccttgtaaggcatttcccccctgactcccttccccgagcctctgcccgggggtcctagcgccgctttctcagccatcccgcctacaacttagccgtccacaacaggatcatctgatcgcgtgcgcccgggctacgatctgcgaggcccgcggaccttgacccggcattgaccgccaccgccccccaggtccgtagggaccaaagaaggggcgggaggaagactgtcacgtggcgccggagttcacgtgactcgtacacatgacttccagtccccgggcgcctcctggagagcaaggacgcgggggagcagagatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggccgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttcaaaaaaaaaaaaaaaaaaaaa

SEQ ID NO: 45 - protein, 481 aa, Homo sapiens
MTSSPRAPPGEQGRGGAEMIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 46 - DNA, 3945 bp, Homo sapiens
ggggcggggccgggagggtacttagggccggggctggcccaggctacggcggctgcagggctccggcaaccgctccggcaacgccaaccgctccgctgcgcgcaggctgggctgcaggctctcggctgcagcgctgggctggtgtgcagtggtgcgaccacggctcacggcagcctcagccacccagatgtaagcgatctggttcccacctcagcctcccgagtagtgtcttcaggcctatggagagcagcttgcgtgggctgggcctgcagtacctggtttgcatagatgattggcaggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 47 - protein, 339 aa, Homo sapiens; identical to SEQ ID NO: 8

SEQ ID NO: 48 - DNA, 3902 bp, Homo sapiens
ggggcggggccgggagggtacttagggccggggctggcccaggctacggcggctgcagggctccggcaaccgctccggcaacgccaaccgctccgctgcgcgcaggctgggctgcaggctctcggctgcagcgctgggtgtcttcaggcctatggagagcagcttgcgtgggctgggcctgcagtacctggtttgcatagatgattggcaggtgggcagcacggggaaggacctgtgagtggccaacctggttcaggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 49 - protein, 339 aa, Homo sapiens; identical to SEQ ID NO: 8

SEQ ID NO: 50 - DNA, 3871 bp, Homo sapiens
ggggcggggccgggagggtacttagggccggggctggcccaggctacggcggctgcagggctccggcaaccgctccggcaacgccaaccgctccgctgcgcgcaggctgggctgcaggctctcggctgcagcgctgggctggtgtgcagtggtgcgaccacggctcacggcagcctcagccacccagatgtaagcgatctggttcccacctcagcctcccgagtagtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 51 - protein, 339 aa, Homo sapiens; identical to SEQ ID NO: 8

SEQ ID NO: 52 - DNA, 3857 bp, Homo sapiens
ggggcggggccgggagggtacttagggccggggctggcccaggctacggcggctgcagggctccggcaaccgctccggcaacgccaaccgctccgctgcgcgcaggctgggctgcaggctctcggctgcagcgctgggtgtcttcaggcctatggagagcagcttgcgtgggctgggcctgcagtacctggtttgcatagatgattggcaggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 53 - protein, 339 aa, Homo sapiens; identical to SEQ ID NO: 8

SEQ ID NO: 54 - DNA, 3982 bp, Homo sapiens
agggccggggctggcccaggctacggcggctgcagggctccggcaaccgctccggcaacgccaaccgctccgctgcgcgcaggctgggctgcaggctctcggctgcagcgctgggctggtgtgcagtggtgcgaccacggctcacggcagcctcagccacccagatgtaagcgatctggttcccacctcagcctcccgagtagatacttctgaaaatagaaatgatgactctgggatgcaaacgttggctgtcctatgtataaggagatggcttttcacgctcccagtgactgaggaagtttctcccagatggcgctgctctgagcctggtgcagggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 55 - protein, 339 aa, Homo sapiens; identical to SEQ ID NO: 8

SEQ ID NO: 56 - DNA, 4086 bp, Homo sapiens
caggaccgccgagggaggcgcctgcgaggaagagctcggccgggtccggagactgctgcctgggaccgcgctcccagcgcctgggcctcggtgtctccgggccaaactgccgacataatcgcatctgccggcatctattttcggtttatttccccctcattgcgaaggatttgcctggccaactttctgcgcaagatcccacgcaattcctgggaccccagaagacaggtcctgttgaagaacaggaatctggcactgggtgggctggggaggaagccgcacggtgttaaatccataaacaggaagagaaaccagacagcgaaaccaagaggcgaatgggcgattggatgccggtggggagaaggccgggggcgcaccctgctcctggactccagtaaagggaggccgggcagagtccctggggcgccacctccccctcggtggatctaggatccggcttccaacatgtggcagctctgggcctccctctgctgcctgctggtgttggccaatgcccggagcaggccctctttccatcccctgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccggatctgcatccacaccaatgcgcacgtcagcgtggaggtgtcggcggaggacctgctcacatgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgtggaatcgaatcagaagtggtggctggaattccacgcaccgatcagtactgggaaaagatctaatctgccgtgggcctgtcgtgccagtcctgggggcgagatcggggtagaaatgcattttattctttaagttcacgtaagatacaagtttcagacagggtctgaaggactggattggccaaacatcagacctgtcttccaaggagaccaagtcctggctacatcccagcctgtggttacagtgcagacaggccatgtgagccaccgctgccagcacagagcgtccttccccctgtagactagtgccgtagggagtacctgctgccccagctgactgtggccccctccgtgatccatccatctccagggagcaagacagagacgcaggaatggaaagcggagttcctaacaggatgaaagttcccccatcagttcccccagtacctccaagcaagtagctttccacatttgtcacagaaatcagaggagagacggtgttgggagccctttggagaacgccagtctcccaggccccctgcatctatcgagtttgcaatgtcacaacctctctgatcttgtgctcagcatgattctttaatagaagttttattttttcgtgcactctgctaatcatgtgggtgagccagtggaacagcgggagacctgtgctagttttacagattgcctccttatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaagttttctccaacttgaagtctactctgatgggatctcagatcctttgtcactgcctatagacttgtagctgctgtctctctttgtccctgcagagaatcacgtcctggaactgcatgttcttgcgactcttgggacttcatcttaacttctcgctgccccagccatgttttcaaccatggcatccctcccccaattagttccctgtcatcctcgtcaaccttctctgtaagtgcctggtaagcttgcccttgcttaagaactcaaaacatagctgtgctctatttttttgttgttgttgtgactgacagagtgagattccgtctcccaggctggagtgcagtggcgccttctcagctcactgcaacctgcagcctcctagattcaagcgattctcctgcttcagccttccgagtagctgggatgacaggcactcaccaatatgcctgggtaatttttgtatttttaagtacatacaggatttcaccatgttggccaggctagtttcaaactcccggcctcaggtggtctgcctgcctcagcctcccaaagtgttgggattacaggcgtgagccactgggccctgcctgtattttttatcagccacaaatccagcaacaagctgaggattcagctcataaaacaggcttggtgtcttggtgatctcacataaccaagatgctaccccgtggggaaccacatccccctggatgccctccagccttggtttgggctggagtcagggcctgtatacagtattttgaatttgtatgccactggtttgcattgctggtcaggaactctagtgctttgcatagccctggtttagaaacatgttatagcagttcttggtatagagcaaactagaagaaccagcaatcattccactgtcctgccaaggtacacctcagtactccccttcccaactgaagtggtatgaggctagctctttccaaaagcattcaagtttggcttctgatgtgactcagaatttaggaaccagatgctagatcaaataagctctgaaaatctgaggaacattgtaggaaaggtttgttaagcatctcttaagtgccatgatgagcataacagccggccgtcgtggctcacgcctgtaatcccagcactttgggaggccaaggtgggaggatgacaaggtcaggagttcaagaccagcctggccaacatgctgaaacctcacctctactaaaaatacaaaaattagctgggcatggtggcacatgcctgtaatcccagctacttgggaggctgaggcaggagaatcgcttgaacccgggaggcggaggttgcagtgagccaagacagtgccagtgcactccagcctcggtgacagcgcaaggctccgtctcaataattaaaaaaaaaaaaaaaaaaaaaaaggccgggcgcagtggctcaagcctgtaatcccagcactttgggaggctgaggcgggcagatcacctgaggtcaggagttttgagatcagccttggcaacacggtgaaaccccatctctactaaaaatacaaaattagccaagcatgctggcacatgcctgtaatcccagctactcgggaggctgaggtacgagaatcgcttgaacctgggaggcagaggatgcagtgagccgagatcacgccattgcactccagcctgggggacaagagtgaatctgtgtctcaccaaaaaaaaaaagaaaaagaaagatgcttaacaaaggttaccataagccacaaattcataaccacttatccttccagtttcaagtagaatatattcataacctcaataaagttctccctgctcccaaa

SEQ ID NO: 57 - protein, 339 aa, Homo sapiens; identical to SEQ ID NO: 8

SEQ ID NO: 58 - DNA, 1587 bp, Homo sapiens
ggcggtgccggccgaacccagacccgaggttttagaagcagagtcaggcgaagctgggccagaaccgcgacctccgcaaccttgagcggcatccgtggagtgcgcctgcgcagctacgaccgcagcaggaaagcgccgccggccaggcccagctgtggccggacagggactggaagagaggacgcggtcgagtaggttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgtagtggtggggcttctttctatttttgatgcactgaatttttgtgtaataaagaacataattgggctctaagccataaaaaaaaaaaaaaaaaaaa

SEQ ID NO: 59 - protein, 333 aa, Homo sapiens [sequence omitted]

SEQ ID NO: 60 - DNA, 1626 bp, Homo sapiens
ggcggtgccggccgaacccagacccgaggttttagaagcagagtcaggcgaagctgggccagaaccgcgacctccgcaaccttgagcggcatccgtggagtgcgcctgcgcagctacgaccgcagcaggaaagcgccgccggccaggcccagctgtggccggacagggactggaagagaggacgcggtcgagtaggtgtgcaccagccctggcaacgagagcgtctaccccgaactctgctggccttgaggttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgtagtggtggggcttctttctatttttgatgcactgaatttttgtgtaataaagaacataattgggctctaagccataaaa

SEQ ID NO: 61 - protein, 333 aa, Homo sapiens [sequence omitted]

SEQ ID NO: 62 - DNA, 1567 bp, Homo sapiens
ggcggtgccggccgaacccagacccgaggttttagaagcagagtcaggcgaagctgggccagaaccgcgacctccgcaaccttgagcggcatccgtggagtgcgcctgcgcagctacgaccgcagcaggaaagcgccgccggccaggcccagctgtggccggacagggactggaagagaggacgcggtcgagttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgtagtggtggggcttctttctatttttgatgcactgaatttttgtgtaataaagaacataattgggctctaagccataaaa

SEQ ID NO: 63 - protein, 333 aa, Homo sapiens [sequence omitted]

SEQ ID NO: 64 - DNA, 1141 bp, Homo sapiens
ggcggtgccggccgaacccagacccgaggttttagaagcagagtcaggcgaagctgggccagaaccgcgacctccgcaaccttgagcggcatccgtggagtgcgcctgcgcagctacgaccgcagcaggaaagcgccgccggccaggcccagctgtggccggacagggactggaagagaggacgcggtcgagtaggttttaaaacatgaatcctacactcatccttgctgccttttgcctgggaattgcctcagctactctaacatttgatcacagtttagaggcacagtggaccaagtggaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgtagtggtggggcttctttctatttttgatgcactgaatttttgtgtaataaagaacataattgggctctaagccataaaa

SEQ ID NO: 65 - protein, 151 aa, Homo sapiens
MDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 66 - DNA, 1401 bp, Homo sapiens
acagctctggacaggctgcttttcattttggtgagtccatccagtacctccacgtgccctgtttttctccaggcacatccttggcctcttccacagtccttgggttttaaaacatgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtgagctggtggacggtgatgaggaaggacttgactggggatggcgcatgcatgggaggaattcatcttcagtctaccagcccccgctgtgtcggatacacactcgaatcattgaagatccgagtgtgatttgaattctgtgatattttcacactggtaaatgttacctctattttaattactgctataaataggtttatattattgattcacttactgactttgcattttcgtttttaaaaggatgtataaatttttacctgtttaaataaaatttaatttcaaatgta

SEQ ID NO: 67 - protein, 333 aa, Homo sapiens [sequence omitted]

SEQ ID NO: 68 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 69 - protein, 401 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSAFATQVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 70 - protein, 396 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 71 - protein, 363 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNMLWSVPTLTSCRMSPSPLTESPIPSAQLPTPYWTSWMECSSAAVAFKDLTSTLQLGPSGSWGMSSFDSFTQSLTVGITVWDWPQQSPKEGPCVCACLSDRP

SEQ ID NO: 72 - protein, 12 aa, Artificial Sequence
  Substrate competitive inhibitor, L803-mts
  MISC_FEATURE (11)..(11): May be N-terminally myristoylated
  MISC_FEATURE (11)..(11): May be a phosphorylated residue
GKEAPPAPPQSP

(19) United States
(12) Patent Application Publication
(10) Pub. No.: US 2019/0183985 A1
(43) Pub. Date: Jun. 20, 2019

(54) METHODS AND COMPOSITIONS FOR THE TREATMENT OF AMYLOIDOSIS

(71) Applicant: Ultragenyx Pharmaceutical Inc., Novato, CA (US)

(72) Inventors: Emil D. KAKKIS, San Rafael, CA (US); Michel Claude VELLARD, San Rafael, CA (US); Andrzej SWISTOWSKI, Petaluma, CA (US)

(21) Appl. No.: 16/226,092

(22) Filed: Dec. 19, 2018

Related U.S. Application Data
(63) Continuation of application No. 15/338,242, filed on Oct. 28, 2016, now abandoned.
(60) Provisional application No. 62/248,713, filed on Oct. 30, 2015.

(51) Int. Cl. A61K 38/48; A61K 35/12; A61K 31/69; A61K 31/675; A61K 31/573; A61K 31/475; A61K 31/454; A61K 31/198; A61K 45/06; A61K 31/704; A61K 38/54; A61K 38/05; C12N 9/64; C12N 9/48

(52) U.S. Cl. CPC A61K 38/488; B82Y 5/00; A61K 38/4813; A61K 31/69; A61K 31/675; A61K 31/573; A61K 31/475; A61K 31/454; A61K 31/198; A61K 45/06; A61K 31/704; A61K 38/54; A61K 38/05; C12N 9/6472; A61K 38/4873; C12N 9/485; C12Y 304/23005; C12N 9/6478; C12Y 304/16001; C12Y 304/22001; A61K 35/12

(57) ABSTRACT

Methods and compositions for the treatment or prevention of amyloidosis are provided. In some embodiments, the methods comprise administering to the subject a therapeutically effective amount of at least one catabolic enzyme or a biologically active fragment thereof. Such methods and compositions may be employed to reduce, prevent, degrade and/or eliminate amyloid formation in the lysosome and/or extracellularly.

### CROSS-REFERENCE TO RELATED APPLICATIONS

This application is a Continuation of U.S. patent application Ser. No. 15/338,242, filed Oct. 28, 2016, which claims priority to U.S. Provisional Application Ser. No. 62/248,713, filed Oct. 30, 2015, each of which is herein incorporated by reference in its entirety for all purposes.

### TECHNICAL FIELD

The present invention relates to compositions and methods suitable for the prevention or treatment of amyloidosis. For instance, catabolic enzymes are provided to reduce, prevent, or eliminate amyloid formation.

### DESCRIPTION OF TEXT FILE SUBMITTED ELECTRONICALLY

The contents of the text file submitted electronically herewith are incorporated herein by reference in their entirety: A computer readable format copy of the Sequence Listing (filename: ULPI_034_02US_SeqList_ST25.txt, date recorded: Dec. 18, 2018, file size: 148 kilobytes).

### BACKGROUND

Amyloids are insoluble fibrous protein aggregates sharing specific structural traits, e.g., a beta-pleated sheet. They arise from at least 18 inappropriately folded versions of proteins and polypeptides present naturally in the body. These misfolded structures alter their proper configuration such that they erroneously interact with one another or other cell components forming insoluble amyloid fibrils. They have been associated with the pathology of more than 20 serious human diseases. Abnormal accumulation of these amyloid fibrils in organs may lead to amyloidosis, and may play a role in various neurodegenerative disorders, as well as other disorders.

The formation of these fibrils involves a passage through the lysosome where the acidic environment allows the formation of the protein aggregates. The amyloids are then released from the cell by exocytosis or by cell lysis.

Trying to eliminate specific fibrils has been the objective of significant research on amyloidosis but without success. Current treatment of amyloidosis involves chemotherapy agents or steroids, such as melphalan and dexamethasone. However, such treatment is not appropriate for all patients and is not effective in many cases due to its specificity. Therefore, there is a great need for alternatives that may safely and effectively prevent or treat diseases associated with amyloidosis.

The present invention solves the problem of how to prevent and stop the formation of excessive amyloids which have a very deleterious activity in the body. The present invention also solves the problem of specificity, and is applicable to different sources of amyloids and not restricted to a specific disease. The present invention also helps the degradation of already formed fibrils by keeping the lysosome more functional and ready to digest fibrils through endocytosis.

### SUMMARY OF THE INVENTION

The present invention provides methods of treating or preventing amyloidosis in a subject. In some embodiments, the methods comprise administering to the subject a composition comprising a therapeutically effective amount of at least one catabolic enzyme or a biologically active fragment thereof.

In some embodiments, the catabolic enzyme is selected from the group consisting of protective protein/cathepsin A (PPCA), neuraminidase 1 (NEU1), tripeptidyl peptidase 1 (TPP1), cathepsin B, cathepsin D, cathepsin E, cathepsin K, and cathepsin L. In some embodiments, the catabolic enzyme acts to prevent the formation of and/or degrade amyloid within the lysosome, i.e., intralysomally. In other embodiments, the catabolic enzyme acts to prevent the formation of and/or degrade amyloid outside the cell, i.e., extracellularly.

In some embodiments, the catabolic enzyme comprises a PPCA polypeptide, or a biologically active fragment thereof. In some embodiments, the PPCA polypeptide comprises an amino acid sequence with at least 85% sequence identity to SEQ ID NO: 2, 43, or 45, or a biologically active fragment thereof. In some embodiments, the PPCA polypeptide comprises the amino acid sequence of SEQ ID NO: 2, 43, or 45, or a biologically active fragment thereof.

In some embodiments, the methods comprise administering a composition comprising a vector, wherein the vector comprises a nucleotide sequence encoding at least one catabolic enzyme of the present invention. In some embodiments, the vector is a viral vector. In some embodiments, the catabolic enzyme is PPCA or a biologically active fragment thereof. In some embodiments, the administration of the PPCA catabolic enzyme comprises administration of a vector encoding a nucleotide sequence having at least 85% identity to SEQ ID NO: 1, 42, or 44. In some embodiments, the nucleotide sequence comprises SEQ ID NO: 1, 42, or 44.

In some embodiments, the catabolic enzyme comprises a NEU1 polypeptide, or a biologically active fragment thereof. In some embodiments, the NEU1 polypeptide comprises an amino acid sequence with at least 85% sequence identity to SEQ ID NO: 4, or a biologically active fragment thereof. In some embodiments, the NEU1 polypeptide comprises the amino acid sequence of SEQ ID NO: 4, or a biologically active fragment thereof.

In some embodiments, the administration of the NEU1 catabolic enzyme comprises administration of a vector encoding a nucleotide sequence having at least 85% identity to SEQ ID NO: 3. In some embodiments, the nucleotide sequence comprises SEQ ID NO: 3.

In some embodiments, the catabolic enzyme comprises a TPP1 polypeptide, or a biologically active fragment thereof. In some embodiments, the TPP1 polypeptide comprises an amino acid sequence with at least 85% sequence identity to SEQ ID NO: 6, or a biologically active fragment thereof. In some embodiments, the TPP1 polypeptide comprises the amino acid sequence of SEQ ID NO: 6, or a biologically active fragment thereof.

In some embodiments, the administration of the TPP1 catabolic enzyme comprises administration of a vector encoding a nucleotide sequence having at least 85% identity to SEQ ID NO: 5. In some embodiments, the nucleotide sequence comprises SEQ ID NO: 5.

In some embodiments, at least two catabolic enzymes are administered to the subject. In some embodiments, the at least two catabolic enzymes are selected from protective protein/cathepsin A (PPCA), neuraminidase 1 (NEU1), tripeptidyl peptidase 1 (TPP1), cathepsin B, cathepsin D, cathepsin E, cathepsin K, and cathepsin L.

In some embodiments, the at least two catabolic enzymes comprise PPCA and NEU1.

In some embodiments, the catabolic enzyme is targeted to the cell lysosome. In other embodiments, the catabolic enzyme is modified to remain outside the cell, i.e., the enzyme is modified to act extracellularly.

In some embodiments, the catabolic enzyme prevents the accumulation of and/or degrades amyloid in the cell lysosome. In other embodiments, the catabolic enzyme prevents the accumulation of and/or degrades amyloid outside the cell, i.e., extracellularly.

In some embodiments, the present invention provides a composition comprising at least two catabolic enzymes, wherein the composition comprises at least one catabolic enzyme that is targeted to the cell lysosome and at least one catabolic enzyme that remains outside the cell. In some embodiments, the catabolic enzymes are selected from protective protein/cathepsin A (PPCA), neuraminidase 1 (NEU1), tripeptidyl peptidase 1 (TPP1), cathepsin B, cathepsin D, cathepsin E, cathepsin K, and cathepsin L. In an exemplary embodiment, the present invention provides a composition comprising at least two catabolic enzymes, wherein the composition comprises a PPCA catabolic enzyme that is targeted to the cell lysosome and a PPCA catabolic enzyme that remains outside the cell.

In some embodiments, the methods further comprise the administration of one or more additional drugs for treating or preventing amyloidosis. In some embodiments, the one or more additional drugs is/are selected from melphalan, dexamethasone, prednisone, bortezomib, lenalidomide, vincristine, doxorubicin, and cyclophosphamide.

In some embodiments, the methods further comprise the administration of one or more drugs that acidifies the lysosome. In some embodiments, the drug that acidifies the lysosome is selected from an acidic nanoparticle, a catecholamine, a β-adrenergic receptor agonist, an adenosine receptor agonist, a dopamine receptor agonist, an activator of the cystic fibrosis transmembrane conductance regulator (CFTR), cyclic adenosine monophosphate (cAMP), a cAMP analog, and an inhibitor of glycogen synthase kinase-3 (GSK-3).

In some embodiments, the methods further comprise the administration of one or more drugs that modulates the lysosome. In an exemplary embodiment, the drug is Z-phenylalanyl-alanyl-diazomethylketone (PADK) or a PADK analog, or a pharmaceutically acceptable salt or ester thereof. In some embodiments, the PADK analog is selected from Z-L-phenylalanyl-D-alanyl-diazomethylketone (PdADK), Z-D-phenylalanyl-L-alanyl-diazomethylketone (dPADK), and Z-D-phenylalanyl-D-alanyl-diazomethylketone (dPdADK).

In some embodiments, the methods further comprise the administration of one or more drugs that promotes autophagy. In an exemplary embodiment, the drug is selected from an activator of peroxisome proliferator-activated receptor gamma coactivator 1-α (PGC-1α), an inhibitor of Lysine (K)-specific demethylase 1A (LSD1), an agonist of Peroxisome proliferator-activated receptor (PPAR), an activator of Transcription factor EB (TFEB), an inhibitor of mechanistic target of rapamycin (mTOR), and an inhibitor of glycogen synthase kinase-3 (GSK3).

In some embodiments, the subject is further treated with stem cell transplantation.

In some embodiments, the administration is parenteral. In some embodiments, the administration is intramuscular, intraperitoneal, or intravenous.

In some embodiments, any one of the compositions and drugs provided herein comprise a pharmaceutically acceptable carrier.

In some embodiments, the subject is a mammal. In some embodiments, the subject is a human.

In some embodiments, the amyloidosis is light-chain (AL) amyloidosis.

In some embodiments, the AL amyloidosis involves one or more organs selected from the heart, the kidneys, the nervous system, and the gastrointestinal tract.

In some embodiments, the amyloidosis is amyloid-beta (Aβ) amyloidosis.

In some embodiments, the Aβ amyloidosis involves one or more organs selected from the brain, the nervous system, and/or involves various muscles, e.g., muscles of the arms and legs. In some embodiments, the Aβ amyloidosis is associated with Alzheimer's disease. In some embodiments, the Aβ amyloidosis is associated with cerebral amyloid angiopathy. In some embodiments, the Aβ amyloidosis is associated with Lewy body dementia. In some embodiments, the Aβ amyloidosis is associated with inclusion body myositis.

### BRIEF DESCRIPTION OF THE DRAWINGS

FIG. 1A-B shows the aggregation of synthetic Aβ42 peptide and Aβ15-36 peptide (negative control) monitored by Thioflavin-T (THT). FIG. 1A. Aggregation at physiological conditions. FIG. 1B. Aggregation at acidic pH.

FIG. 2A-B shows the aggregation of synthetic Aβ42 peptide in vitro over a 24 hour time period as detected by western blot. FIG. 2A. 12% Bis-Tris gel, reducing conditions, probed with 6E10, a commercially available purified anti-3-amyloid antibody that is reactive to amino acid residues 1-16 of beta amyloid. FIG. 2B. 18% Tris-Glycine gel, reducing conditions, probed with 6E10.

FIG. 3A-D show that cathepsin A (interchangeably referred to herein as Cath A or PPCA) prevents the aggregation of Aβ42 amyloid species. FIG. 3A. Activation of 90 ng cathepsin A by cathepsin L (full black circles). FIG. 3B. Activation of 450 ng cathepsin A by cathepsin L. FIG. 3C. Preventive effect of 90 ng PPCA on Aβ42 aggregation and the inhibition of PPCA by the serine protease inhibitor, PMSF (phenylmethylsulfonyl fluoride). FIG. 3D. Preventive effect of 450 ng PPCA on Aβ42 aggregation. Aβ42 peptides were aggregated alone (open circles), with two concentrations of Cath A (open squares) and with combination of Cath A+inhibitor PMSF (open triangles). Cath A only (full squares) and inhibitor PMSF only (full triangles) were incubated with THT reagent and served as negative controls.

FIG. 4A-B shows that Cath A (i.e., PPCA) prevents the aggregation of Aβ42 amyloid species in a dose-dependent manner. FIG. 4A. Graph showing Aβ42 aggregation over 2 hours at pH5, 37° C. with varying PPCA concentrations (7 ng to 900 ng) as measured by THT. Aβ42 aggregation was measured alone and with serial dilutions of PPCA. Lines are labeled for clarity. FIG. 4B. Bar graph showing end-point (2 hrs) Aβ42 aggregation.

FIG. 5 shows that Cath A (i.e., PPCA) prevents the aggregation of both high and lower molecular weight species of Aβ42 amyloid. Treatment of 0.9 μg Aβ42 monomer with 500 ng PPCA is shown over a time period of 2 hours on an 18% Tris-Glycine gel, under reducing conditions, probed with 6E10.

FIG. 6A-D show that cathepsin B (Cath B) prevents the aggregation of Aβ42 amyloid. FIG. 6A. Activation of 90 ng cathepsin B and its inhibition by the protease inhibitor E64. FIG. 6B. Activation of 450 ng cathepsin B and its inhibition by E64. FIG. 6C. Preventive effect of 90 ng cathepsin B on Aβ42 aggregation and the lack inhibition by E64. FIG. 6D. Preventive effect of 450 ng cathepsin B on Aβ42 aggregation and the lack inhibition by E64. Aβ42 peptides were aggregated alone (open circles), with two concentrations of Cath B (open squares) and with combination of Cath B+inhibitor E64 (open triangles). Cath B only (full squares) and inhibitor E64 only (full triangles) were incubated with THT reagent and served as negative controls.

FIG. 7A-B shows that cathepsin B moderately prevents the aggregation of Aβ42 amyloid species in a dose-dependent manner. FIG. 7A. Graph showing Aβ42 aggregation over 2 hours at pH5, 37° C. with varying cathepsin B concentrations (7 ng to 900 ng) as measured by THT. Aβ42 aggregation was measured alone and with serial dilutions of cathepsin B. FIG. 7B. Bar graph showing end-point (2 hrs) Aβ42 aggregation.

FIG. 8 shows that cathepsin B prevents the aggregation of both low molecular weight species of Aβ42 amyloid and degrades Aβ42 in a time dependent manner. Treatment of 0.9 μg Aβ42 monomer with 200 ng cathepsin B is shown over a time period of 2 hours on an 18% Tris-Glycine gel, under reducing conditions, probed with 6E10

FIG. 9 shows that cathepsin D prevents the aggregation of Aβ42 amyloid as monitored by THT. Aβ42 peptides were aggregated alone (empty circles) and with cathepsin D (empty squares) over period of 2 hours. Cathepsin D alone (triangles) was incubated with THT reagent and served as a negative control.

FIG. 10 shows a western blot demonstrating that PPCA, cathepsin B, PPCA plus cathepsin B, and cathepsin D degrade high molecular weight oligomers/fibrils of Aβ42 amyloid. Cathepsin D degrades low molecular oligomers and completely eliminates Aβ42 monomers.

FIG. 11 shows a western blot demonstrating a comparison in the detection of Aβ42 oligomers and fibrils using an oligomer specific A11 antibody. Aβ42 peptides were subjected to 7 day aggregation protocols specific for oligomers and fibrils. Reduction of oligomer form in fibril formation (line 9) indicates transition of oligomers into fibril form, which is not detected by oligomer specific A11 antibody.

FIG. 12 shows a western blot demonstrating a comparison in the detection of Aβ42 oligomers and fibrils using an oligomer and fibril specific E610 antibody. Aβ42 peptides were subjected to 7 day aggregation protocols specific for oligomers and fibrils. Fibril formation was not detected in the oligomer specific protocol at day 7 (line 4). Reduction of oligomer form and appearance of fibril form (smear on line 9) was detected in the fibril formation protocol.

FIG. 13 shows a western blot illustrating the enzymatic degradation of Aβ42 oligomers as probed by the oligomer specific A11 antibody. Lines 1-6 contain day 9 oligomers aggregated at pH 7.0 at 25° C. and additionally treated overnight at 37° C. in enzyme specific pH. Lines 1-3 are not treated with enzymes. Lines 4-6 represent treatment with 90 ng of cathepsin A, B, and D, respectively. Line 8 contains day 9 oligomers aggregated at pH 7.0 at 25° C. Line 9 contains monomers at pH 7.0. Degradation of oligomers by 90 ng of cathepsin A is shown in line 4. 2 μg of material was loaded on each line.

FIG. 14 shows a western blot illustrating the enzymatic degradation of Aβ42 fibrils as probed by the oligomer and fibril specific antibody E610. Lines 1-6 contain day 9 fibrils aggregated at pH 7.0 at 25° C. and additionally treated overnight at 37° C. in enzyme specific pH. Lines 1-3 are not treated with enzymes. Lines 4-6 represent treatment with 90 ng of cathepsin A, B, and D, respectively. Line 8 contains day 9 fibers aggregated at pH 7.0 at 25° C. Line 9 contains monomers at pH 7.0. Degradation of fibers and oligomers by 90 ng of cathepsin A is shown in line 4. Degradation of fibers by 90 ng of cathepsin B is shown in line 5. 2 μg of material was loaded on each line.

FIG. 15 shows a human Aβ42 specific ELISA used to monitor the degradation of Aβ42 monomers with cathepsin A. Treatment of Aβ42 monomers with 90 ng of cathepsin A (striped bars) showed degradation from the C-terminus at various time points (0, 10, 30, 60, 120 min), which is reflected in loss of C-terminal capture by capturing antibody and in effect loss of fluorescent signal. In contrast, Aβ42 monomers not treated with cathepsin A showed lack of C-terminal degradation (solid bars), which is reflected in efficient antibody capture and strong fluorescent signal. An inhibitor of amyloid aggregation, phenol red was used in both cases to prevent peptide aggregation, which could affect capture by the C-terminal antibody in ELISA.

FIG. 16A-B show aggregation of Aβ40 and Aβ42 measured by THT assay. Aβ40, Aβ42, and Aβ16 were co-incubated with ThT for 2 h at 37° C. to measure the kinetics of aggregation. Aβ42 aggregates more efficiently and faster than Aβ40. FIG. 16A. Graphical representation aggregation of Aβ peptides on a single scale. FIG. 16B. Graphical representation of Aβ40 aggregation on a separate scale.

FIG. 17A-C show that simultaneous incubation of Aβ40, Cath A, and THT shows no change in Aβ40 aggregation. Increasing concentrations of Cath A were co-incubated with 15 μM Aβ40 and 2 mM ThT for 2 h at 37° C. to measure how Cath A affected the kinetics of Aβ40 aggregation. FIG. 17A. 900 ng Cath A was co-incubated with Aβ40 and THT. FIG. 17B. 1000 ng Cath A was co-incubated with Aβ40 and THT. FIG. 17C. 2250 ng Cath A was co-incubated with Aβ40 and THT.

FIG. 18A-C show that Aβ40 pre-incubated with Cath A leads to loss of its aggregation potential as revealed by lack of THT fluorescence. Aβ40 and 2500 ng Cath A were first incubated for 30′, 1 h, and 2 h at 37° C. (FIGS. 18A, 18B, and 18C, respectively). Reactions were then co-incubated with ThT for 2 h at 37° C. to measure how Cath A affected the kinetics of Aβ40 aggregation.

FIG. 19A-B show detection of cleavage of Aβ40 C-terminal end using a C-terminal capture antibody. Aβ40 peptide was incubated for 2 h at 37° C. at pH5 with varying concentrations of Cath A. The reaction was transferred to an ELISA plate pre-coated with a C-terminal capture antibody and was co-incubated with N-terminal detection antibody overnight at 4° C. Error bars are referring to the standard deviation in the OD values. FIG. 19A. Recovery rate of undigested Aβ40 in samples treated with increased concentrations of Cath A. FIG. 19B. Mean absorbance at 450 nm of samples in ELISA wells treated with increased concentrations of Cath A.

FIG. 20A-C show aggregation and degradation of Aβ40 amyloid measured by Western Blot. FIG. 20A. Aggregation into amyloid species. Aβ40 was incubated in either Fibril Buffer or Oligomer buffer at RT for 0-9 days. 2 μg of Aβ40 were loaded per lane on an 18% Tris-Glycine gel and transferred to a PVDF membrane. The blot was probed with an Anti-Aβ40 C-terminal primary antibody (G2-10). Aβ40 incubated with Cath A during fibril formation prevents aggregation. Aβ40 was co-incubated with Cath A in fibril buffer at RT for 0-9 days. To observe high molecular weight bands the gel in FIG. 20B was run on a 7.5% Tris-glycine gel and to see the low molecular weight bands gel in FIG. 20C was run on an 18% Tris-glycine gel. 2 μg of Aβ40 were loaded into each lane. Each gel was transferred to a PVDF membrane and probed with an Anti-Aβ40 C-terminal primary antibody (G2-10).

### DETAILED DESCRIPTION

As shown herein, the present inventors have discovered that various catabolic enzymes can be used to prevent the formation of and/or degrade various types of amyloid oligomers and fibrils. Because these oligomers and fibrils can contribute to the development of a variety of amyloid-associated diseases and disorders, the present invention is directed to methods and compositions for the treatment or prevention of amyloidosis in a subject.

Amyloids are insoluble fibrous protein aggregates sharing specific structural traits. The deposition of normally soluble proteins in this insoluble form can lead to cell death and tissue degeneration. To date, 18 different proteins and polypeptides have been identified in disease-associated amyloid deposits. See Westermark et al. (“Nomenclature of amyloid fibril proteins. Report from the meeting of the International Nomenclature Committee on Amyloidosis, Aug. 8-9, 1998. Part 1.” Amyloid. 1999 March; 6(1):63-6), which is incorporated by reference in its entirety. The amyloid fibrils are long, straight, unbranched filaments about 40-120 Å in diameter, which bind to physiological dyes such as Congo red and thioflavine T and are resistant to protease digestion.

As used herein, amyloidosis refers to a disease that results from accumulation of amyloids. Such diseases to be treated or prevented by the present invention include, but are not limited to, systemic AL amyloidosis, Alzheimer's Disease, Diabetes mellitus type 2, Parkinson's disease, Transmissible spongiform encephalopathy e.g. Bovine spongiform encephalopathy, Fatal Familial Insomnia, Huntington's Disease, Medullary carcinoma of the thyroid, Cardiac arrhythmias, Atherosclerosis, Rheumatoid arthritis, Aortic medial amyloid, Prolactinomas, Familial amyloid polyneuropathy, Hereditary non-neuropathic systemic amyloidosis, Dialysis related amyloidosis, Finnish amyloidosis, Lattice corneal dystrophy, Cerebral amyloid angiopathy, Cerebral amyloid angiopathy (Icelandic type), Sporadic Inclusion Body Myositis, Amyotrophic lateral sclerosis (ALS), Prion-related or Spongiform encephalopathies, such as Creutzfeld-Jacob, Dementia with Lewy bodies, Frontotemporal dementia with Parkinsonism, Spinocerebellar ataxias, Spinocerebellar ataxia, Spinal and bulbar muscular atrophy, Hereditary dentatorubral-pallidoluysian atrophy, Familial British dementia, Familial Danish dementia, Non-neuropathic localized diseases, such as in Type II diabetes mellitus, Medullary carcinoma of the thyroid, Atrial amyloidosis, Hereditary cerebral haemorrhage with amyloidosis, Pituitary prolactinoma, Injection-localized amyloidosis, Aortic medial amyloidosis, Hereditary lattice corneal dystrophy, Corneal amyloidosis associated with trichiasis, Cataract, Calcifying epithelial odontogenic tumors, Pulmonary alveolar proteinosis, Inclusion-body myositis, Cutaneous lichen amyloidosis, and Non-neuropathic systemic amyloidosis, such as AL amyloidosis, AA amyloidosis, Familial Mediterranean fever, Senile systemic amyloidosis, Familial amyloidotic polyneuropathy, Hemodialysis-related amyloidosis, ApoAI amyloidosis, ApoAII amyloidosis, ApoAIV amyloidosis, Finnish hereditary amyloidosis, Lysozyme amyloidosis, Fibrinogen amyloidosis, Icelandic hereditary cerebral amyloid angiopathy, familial amyloidosis, and systemic amyloidosis which occurs in multiple tissues, such as light-chain amyloidosis, and other various neurodegenerative disorders. In exemplary embodiments, the amyloidosis is light-chain (AL) amyloidosis. In further exemplary embodiments, the AL amyloidosis involves one or more organs selected from the heart, the kidneys, the nervous system, and the gastrointestinal tract.

In some embodiments, the present invention provides methods and compositions for the treatment or prevention of a disease associated with amyloidosis in a subject, wherein the disease is associated with the formation of amyloid-beta (Aβ or Abeta) peptides. These peptides result from the amyloid precursor protein (APP), which is cleaved by beta secretase and gamma secretase to yield amyloid-beta. In some embodiments, the disease associated with the formation of amyloid-beta is selected from Alzheimer's Disease, cerebral amyloid angiopathy, Lewy body dementia, and inclusion body myositis.

In alternative embodiments, the present invention provides methods and compositions for the treatment or prevention of a disease associated with amyloidosis in a subject, wherein the disease is not associated with the formation of amyloid beta, i.e., wherein the disease is a disease other than one associated with the formation of amyloid beta, e.g., a disease other than Alzheimer's disease, cerebral amyloid angiopathy, Lewy body dementia, and inclusion body myositis.

In one embodiment, the disease associated with amyloidosis is light-chain (AL) amyloidosis. In another embodiment, the disease associated with amyloidosis is selected from Parkinson's Disease, Huntington's Disease, Rheumatoid arthritis, and a prion-related disease.

In some embodiments, the amyloidosis is a systemic amyloidosis. Systemic amyloidosis encompasses a complex group of diseases caused by tissue deposition of misfolded proteins that result in progressive organ damage.

As noted above, in some embodiments, the amyloidosis is light-chain (AL) amyloidosis (also known as, i.e. a.k.a., primary systemic amyloidosis (PSA) or primary amyloidosis). AL amyloidosis refers to a condition caused when a subject's antibody-producing cells do not function properly and produce abnormal protein fibers made of components of antibodies called light chains. In some embodiments, such light chains form amyloid deposits in one or more different organs which may cause or already caused damage to these organs. In some embodiments, the abnormal light chains are in blood and/or urine. In some embodiments, the abnormal light chains are “Bence Jones proteins”. In some embodiments, the AL amyloidosis affects the heart, peripheral nervous system, gastrointestinal tract, blood, lungs and/or skin. Clinical features of AL amyloidosis also may include a constellation of symptoms and organ dysfunction that can include cardiac, renal, and hepatic dysfunction, gastrointestinal involvement, neuropathies and macroglossia.

In some embodiments, the amyloidosis is AA amyloidosis (a.k.a. secondary amyloidosis, AA), caused by deposited proteins called serum amyloid A protein (SAA). In some embodiments, the SAA protein is mainly deposited in the liver, spleen and/or kidney. In some embodiments, the AA amyloidosis leads to nephrotic syndrome. In some embodiments, the AA amyloidosis is caused by autoimmune diseases (e.g., Rheumatoid arthritis, Ankylosing spondylitis, or Crohn's disease and ulcerative colitis), Chronic infections (e.g., Tuberculosis, Bronchiectasis, or Chronic osteomyelitis), autoinflammatory diseases (e.g., Familial Mediterranean fever (FMF), Muckle-Wells syndrome (MWS), Cancer (e.g., Hodgkin's lymphoma, Renal cell carcinoma), and/or Chronic foreign body reaction (e.g., Silicone-induced granulomatous reaction).

In some embodiments, the amyloidosis is familial amyloidosis. In some embodiments, the familial amyloidosis is ATTR amyloidosis (a.k.a. or senile systemic amyloidosis) which is due one or more inherited amyloidosis, such as a mutation in the transthyretin (TTR) gene that produces abnormal transthyretin protein. In some embodiments, the familial amyloidosis is caused by one or more mutation in apolipoprotein A-I (AApoAI), apolipoprotein A-II (AApoAII), gelsolin (AGel), fibrinogen (AFib), lysozyme (ALys), and/or Lect2.

In some embodiments, the amyloidosis is Beta-2 Microglobulin Amyloidosis (Abeta2m). Beta-2 microglobulin amyloidosis is caused by chronic renal failure and often occurs in patients who are on dialysis for many years. Amyloid deposits are made of the beta-2 microglobulin protein that accumulated in tissues, particularly around joints, when it cannot be excreted by the kidney because of renal failure.

In some embodiments, the amyloidosis is Localized Amyloidosis (ALoc). In some embodiments, localized amyloid deposits in the airway (trachea or bronchus), eye, or urinary bladder. In some embodiments, the ALoc is caused by local production of immunoglobulin light chains not originating in the bone marrow. In some embodiments, the ALoc is associated with endocrine proteins, or proteins produced in the skin, heart, and other sites. These usually do not become systemic.

In some embodiments, the amyloidosis occurs in the kidney of the subject. In some embodiments, the amyloidosis in the kidney is AA amyloidosis. In some embodiments, the AA amyloidosis leads to nephrotic syndrome. In some embodiments, the amyloidosis in the kidney is AL amyloidosis. In some embodiments, symptoms of kidney disease and renal failure associated with AL amyloidosis include, but are not limited to, fluid retention, swelling, and shortness of breath.

In some embodiments, the amyloidosis occurs in the heart of the subject. In some embodiments, the amyloidosis in the heart is AL amyloidosis. In some embodiments, the amyloidosis in the heart leads to heart failure and/or irregular heart beat.

In some embodiments, the amyloidosis occurs in the gastrointestinal tract of the subject. In some embodiments, symptoms of GI amyloidosis include, but are not limited to, esophageal reflux, constipation, nausea, abdominal pain, diarrhea, weight loss, and early satiety. In some embodiments, the amyloidosis occurs in the duodenum, stomach, colo-rectum, and/or esophagus.

In some embodiments, the treatment methods provided herein alleviate, reduce the severity of, or reduce the occurrence of, one or more of the symptoms associated with amyloidosis. Such symptoms include those symptoms associated with light-chain (AL) amyloidosis (primary systemic amyloidosis) and/or AA amyloidosis (secondary amyloidosis). In some embodiments, the symptoms include, but are not limited to, fluid retention, swelling, shortness of breath, fatigue, irregular heartbeat, numbness of hands and feet, rash, shortness of breath, swallowing difficulties, swollen arms or legs, esophageal reflux, constipation, nausea, abdominal pain, diarrhea, early satiety, stroke, gastrointestinal disorders, enlarged liver, diminished spleen function, diminished function of the adrenal and other endocrine glands, skin color change or growths, lung problems, bleeding and bruising problems, fatigue and weight loss, decreased urine output, diarrhea, hoarseness or changing voice, joint pain, and weakness. In some embodiments, the symptoms are those associated with amyloid-beta (Aβ) amyloidosis. In some embodiments, the symptoms include, but are not limited to, common symptoms of Alzheimer's disease, including memory loss, confusion, trouble understanding visual images and spatial relationships, and problems speaking or writing.

According to the methods of the present invention, the term “subject,” includes any subject that has, is suspected of having, or is at risk for having a disease or condition. Suitable subjects (or patients) include mammals, such as laboratory animals (e.g., mouse, rat, rabbit, guinea pig), farm animals, and domestic animals or pets (e.g., cat, dog). Non-human primates and human patients are also included. A subject “at risk” may or may not have detectable disease, and may or may not have displayed detectable disease prior to the prevention or treatment methods described herein. “At risk” denotes that a subject has one or more so-called risk factors, which are measurable parameters that correlate with development of any one of the diseases, disorders, conditions, or symptoms described herein. A subject having one or more of these risk factors has a higher probability of developing any one of the diseases, disorders, conditions, or symptoms described herein than a subject without these risk factor(s). In some embodiments, the subject is a mammal. In some embodiments, the subject is a human. In some embodiments, the subject is a human diagnosed as having amyloidosis or disease/symptom caused by or associated with amyloidosis. In some embodiments, the subject is a human suspected to have amyloidosis. In some embodiments, the subject is a human having high risk of developing amyloidosis. In some embodiments, the subject is an amyloidosis patient with one or more diseases/conditions/symptoms as described herein.

The terms “treating” and “treatment” as used herein refer to an approach for obtaining beneficial or desired results including clinical results, and may include even minimal changes or improvements in one or more measurable markers of the disease or condition being treated. A treatment is usually effective to reduce at least one symptom of a condition, disease, disorder, injury or damage. Exemplary markers of clinical improvement will be apparent to persons skilled in the art. Examples include, but are not limited to, one or more of the following: decreasing the severity and/or frequency one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life, etc.

“Prophylaxis,” “prophylactic treatment,” “prevention,” or “preventive treatment” refers to preventing or reducing the occurrence or severity of one or more symptoms and/or their underlying cause, for example, prevention of a disease or condition in a subject susceptible to developing a disease or condition (e.g., at a higher risk, as a result of genetic predisposition, environmental factors, predisposing diseases or disorders, or the like).

The present invention provides methods of treating or preventing amyloidosis in a subject. In some embodiments, the methods comprise administering to the subject a composition comprising a therapeutically effective amount of at least one catabolic enzyme or a biologically active fragment thereof. In some embodiments, the methods comprise increasing the expression, activity, and/or concentration of at least one catabolic enzyme in the subject. Increasing the expression, activity, and/or concentration of a given catabolic enzyme may be accomplished at the genomic DNA level, transcriptional level, post-transcriptional level, translational level, and/or post-translational level, including but not limited to, increasing the gene copy number, mRNA transcription rate, mRNA abundance, mRNA stability, protein translation rate, protein stability, protein modification, protein activity, protein complex activity, etc. Increasing the concentration of a given catabolic enzyme may further be accomplished by administering to the subject a composition comprising a therapeutically effective amount of at least one catabolic enzyme or a biologically active fragment thereof. As used herein, the term catabolic enzyme refers not only to the natural form the enzyme, but also any purified, isolated, synthetic, recombinant, and functional variants, fragments, chimeras, and mutants of the natural enzyme.

In some embodiments, the at least one catabolic enzyme is selected from the non-limiting group consisting of protective protein/cathepsin A (PPCA), neuraminidase 1 (NEU1), tripeptidyl peptidase 1 (TPP1), cathepsin B, cathepsin D, cathepsin E, cathepsin K, and cathepsin L.

In some embodiments, the at least one catabolic enzyme is PPCA (a.k.a. Protective Protein Cathepsin A, PPGB, Carboxypeptidase C, EC 3.4.16.5, GSL, GLB2, Carboxypeptidase Y-Like Kininase, NGBE, carboxypeptidase-L, Protective Protein For Beta-Galactosidase (Galactosialidosis), deamidase, Beta-Galactosidase, Lysosomal Carboxypeptidase A, Beta-Galactosidase Protective Protein, Lysosomal Protective Protein, Protective Protein For Beta-Galactosidase, Urinary Kininase, EC 3.4.168, or Carboxypeptidase L) is classified both as a cathepsin and a carboxypeptidase.

In some embodiments, the at least one catabolic enzyme is PPCA. PPCA is a glycoprotein that associates with the lysosomal enzymes beta-galactosidase and neuraminidase to form a complex of high-molecular-weight multimers. The formation of this complex provides a protective role for stability and activity. It is protective for β-galactosidase and neuraminidase. In some embodiments, the PPCA can be a natural, synthetic, or recombinant protein. In some embodiments, the PPCA polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2, 43, or 45. In some embodiments, the PPCA polypeptide comprises the amino acid sequence of SEQ ID NO: 2, 43, or 45.

In some embodiments, the at least one catabolic enzyme is Neuraminidase 1 (NEU1, a.k.a. sialidase 1, lysosomal sialidase, EC 3.2.1.18, Acetylneuraminyl Hydrolase, SIAL1, Lysosomal Sialidase, exo-alpha-sialidase, NANH, sialidase-1, or G9 Sialidase) is a lysosomal neuraminidase enzyme. NEU1 is an enzyme that cleaves terminal sialic acid residues from substrates such as glycoproteins and glycolipids. In some embodiments, the NEU1 can be a natural, synthetic, or recombinant protein. In some embodiments, the NEU1 polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 4. In some embodiments, the NEU1 polypeptide comprises the amino acid sequence of SEQ ID NO: 4.

In some embodiments, the at least one catabolic enzyme is Tripeptidyl peptidase 1 (TPP1, Spinocerebellar Ataxia, Autosomal Recessive 7, CLN2, SCAR7, Growth-Inhibiting Protein 1, Cell Growth-Inhibiting Gene 1 Protein, Lysosomal Pepstatin Insensitive Protease, Tripeptidyl Aminopeptidase, Tripeptidyl-Peptidase 1, LPIC, Lysosomal Pepstatin-Insensitive Protease, or EC 3.4.14.9). TPP1 is an enzyme that cleaves N-terminal tripeptides from substrates and has weaker endopeptidase activity. In some embodiments, the TPP1 can be a natural, synthetic, or recombinant protein. In some embodiments, the TPP1 polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 6. In some embodiments, the TPP1 polypeptide comprises the amino acid sequence of SEQ ID NO: 6.

In some embodiments, the at least one catabolic enzyme is Cathepsin B (a.k.a. EC 3.4.22.1, CPSB, Amyloid Precursor Protein Secretase, Cysteine Protease, APPS, APP secretase, or EC 3.4.22). Cathepsin B is a lysosomal cysteine protease composed of a dimer of disulfide-linked heavy and light chains, both produced from a single protein precursor. In some embodiments, the Cathepsin B can be a natural, synthetic, or recombinant protein. In some embodiments, the Cathepsin B polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 8, 47, 49, 51, 53, 55, or 57. In some embodiments, the Cathepsin B polypeptide comprises the amino acid sequence of SEQ ID NO: 8, 47, 49, 51, 53, 55, or 57.

In some embodiments, the at least one catabolic enzyme is Cathepsin D (a.k.a. EC 3.4.23.5, CTSD). Cathepsin D refers is a lysosomal acid protease active in intracellular protein breakdown. In some embodiments, the Cathepsin D can be a natural, synthetic, or recombinant protein. In some embodiments, the Cathepsin D polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 68. In some embodiments, the Cathepsin D polypeptide comprises the amino acid sequence of SEQ ID NO: 68. In some embodiments, the Cathepsin D polypeptide harbors one or more modifications relative to the amino acid sequence of SEQ ID NO: 68. In certain embodiments, the Cathepsin D polypeptide comprises the amino acid sequence of SEQ ID NO: 68, wherein the polypeptide harbors a modification at an amino acid position selected from position 58 (A to V), position 229 (F to I), position 282 (G to R), and position 383 (W to C).

In some embodiments, the at least one catabolic enzyme is Cathepsin E (a.k.a. EC 3.4.23.34, CTSE). Cathepsin E is a lysosomal aspartyl protease. In some embodiments, the Cathepsin E can be a natural, synthetic, or recombinant protein. In some embodiments, the Cathepsin E polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 69, 70, or 71. In some embodiments, the Cathepsin E polypeptide comprises the amino acid sequence of SEQ ID NO: 69, 70, or 71. In some embodiments, the Cathepsin E polypeptide harbors one or more modifications relative to the amino acid sequence of SEQ ID NO: 69, 70, or 71. In certain embodiments, the Cathepsin E polypeptide comprises the amino acid sequence of SEQ ID NO: 69, wherein the polypeptide harbors a modification at an amino acid position selected from position 82 (I to V) and position 329 (T to I).

In some embodiments, the at least one catabolic enzyme is Cathepsin K (a.k.a. EC 3.4.22.38, CTSO, Pycnodysostosis, PYCD, Cathepsis O, PKND, Cathepsin X). Cathepsin K is a lysosomal cysteine protease involved in bone remodeling and resorption, defined by its high specificity for kinins. In some embodiments, the Cathepsin K can be a natural, synthetic, or recombinant protein. In some embodiments, the Cathepsin K polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 10. In some embodiments, the Cathepsin K polypeptide comprises the amino acid sequence of SEQ ID NO: 10.

In some embodiments, the at least one catabolic enzyme is Cathepsin L (a.k.a. MEP, CTSL, EC 3.4.22.15, CATL, Major Excreted Protein). Cathepsin L is a lysosomal endopeptidase enzyme which is involved in the initiation of protein degradation. Its substrates include collagen and elastin, as well as alpha-1 protease inhibitor, a major controlling element of neutrophil elastase activity. In some embodiments, the Cathepsin L can be a natural, synthetic, or recombinant protein. In some embodiments, the Cathepsin L polypeptide comprises an amino acid sequence with at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 12, 59, 61, 63, 65, or 67. In some embodiments, the Cathepsin L polypeptide comprises the amino acid sequence of SEQ ID NO: 12, 59, 61, 63, 65, or 67.

In some embodiments, the administration comprises the administration of a nucleotide sequence encoding at least one catabolic enzyme of the present invention.

As used herein, the terms “polynucleotide”, “polynucleotide sequence”, “nucleic acid sequence”, “nucleic acid fragment”, “nucleotide sequence,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “chimeric” or “recombinant” when describing a nucleic acid sequence or a protein sequence refers to a nucleic acid or a protein sequence that links at least two heterologous polynucleotides or two heterologous polypeptides into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence. It is recognized that a genetic regulatory element of the present invention comprises a synthetic nucleotide sequence. In some embodiments, the synthetic nucleotide sequence shares little or no extended homology to natural sequences. Extended homology in this context generally refers to 100% sequence identity extending beyond about 25 nucleotides of contiguous sequence. A synthetic genetic regulatory element of the present invention comprises a synthetic nucleotide sequence.

As used herein, an “isolated” or “purified” nucleic acid molecule or polynucleotide, or biologically active portion thereof, is substantially or essentially free from components that normally accompany or interact with the nucleic acid molecule or polynucleotide as found in its naturally occurring environment. Thus, an isolated or purified nucleic acid molecule or polynucleotide is substantially free of other cellular material or culture medium when produced by recombinant techniques, or substantially free of chemical precursors or other chemicals when chemically synthesized.

In some embodiments, the methods comprise administering to the subject a composition comprising an expression vector (interchangeably referred to herein as a vector), wherein the vector comprises a polynucleotide sequence encoding at least one catabolic enzyme. In some embodiments, the methods comprise administering to the subject a composition comprising at least one expression vector comprising an expression cassette of coding genes.

In some embodiments, the expression vector is a viral vector. Accordingly, in the some embodiments, the methods of the present invention comprise administering to the subject a composition comprising at least one viral vector comprising a polynucleotide sequence encoding at least one catabolic enzyme. In some embodiments, the expression cassette, the expression vector, or the viral vector further comprises one or more nucleotide sequences encoding a signal peptide. In some embodiments, the signal peptide is an intralysosomal localization peptide.

A nucleotide sequence encoding at least one catabolic enzyme can be delivered to a subject through any suitable delivery system, such as those described by Rolland (Pharmaceutical Gene Delivery Systems, ISBN: 978-0-8247-4235-5, 2003), which is incorporated by reference in its entirety. In some embodiments, the delivery system is a viral system, a physical system, and/or a chemical system.

In some embodiments, the delivery system to deliver a nucleotide sequence encoding at least one catabolic enzyme is a viral system. In some embodiments, an adenovirus vector is used (see, Thrasher et al., Gene therapy: X-SCID transgene leukaemologenicity. Nature. 2006; 443(7109): E5-E6; Zhang et al., Adenoviral and adeno-associated viral vectors-mediated neuronal gene transfer to cardiovascular control regions of the rat brain. Int J Med Sci. 2013; 10(5): 607-616.). In some embodiments, an adeno-associated vector is used (see, Teramato et al., Crisis of adenoviruses in human gene therapy. Lancet. 2000; 355(9218): 1911-1912, Okada et al., Gene transfer targeting mouse vestibule using adenovirus and adeno-associated virus vectors. Otol Neurotol. 2012; 33(4): 655-659.). In some embodiments, a retroviral vector is used (see, Anson et al., The use of retroviral vectors for gene therapy-what are the risks? A review of retroviral pathogenesis and its relevance to retroviral vector-mediated gene delivery. Genet Vaccines Ther. 2004; 2(1): 9; Frederic D. Retroviral integration and human gene therapy. J Clin Invest. 2007; 117(8): 2083-2086.). In some embodiments, a lentivirus vector is used (see, Goss et al., Antinociceptive effect of a genomic herpes simplex virus-based vector expressing human proenkephalin in rat dorsal root ganglion. Gene Ther. 2001; 8(7): 551-556; Real et al., Improvement of lentiviral transfer vectors using cis-acting regulatory elements for increased gene expression. Appl Microbiol Biotechnol. 2011; 91(6): 1581-91.). In some embodiments, a herpes simplex virus vector is used (see, Lachmann R H, Efstathiou S. The use of herpes simplex virus-based vectors for gene delivery to the nervous system. Mol Med Today. 1997; 3(9): 404-411; Liu S, Dai M, You L, Zhao Y. Advance in herpes simplex viruses for cancer therapy. Sci China Life Sci. 2013; 56(4): 298-305.). In some embodiments, a poxvirus vector is used (see, Moss B. Reflections on the early development of poxvirus vectors. Vaccine. 2013; 31(39): 4220-4222.). Each of the references is incorporated herein by reference in its entirety.

In some embodiments, the delivery system to deliver a nucleotide sequence encoding at least one catabolic enzyme of the invention is a physical system. In some embodiments, the physical systems include, but are not limited to jet injection, biolistics, electroporation, hydrodynamic injection, and ultrasound (see, Sirsi et al. Advances in ultrasound mediated gene therapy using microbubble contrast agents. Theranostics. 2012; 2(12): 1208-1222; Naldini et al., In vivo gene delivery and stable transduction of nondividing cells by a lentiviral vector. Science. 1996; 272(5259): 263-267; Panje et al., Ultrasound-mediated gene delivery with cationic versus neutral microbubbles: Effect of DNA and microbubble dose on in vivo transfection efficiency. Theranostics. 2012; 2(11): 1078-1091; Gao et al., Cationic liposome-mediated gene transfer. Gene Ther. 1995; 2(10): 710-722; Orio et al., Electric field orientation for gene delivery using high-voltage and low-voltage pulses. J Membr Biol. 2012; 245(10): 661-666.) Each of the references is incorporated herein by reference in its entirety.

In some embodiments, the delivery system to deliver a nucleotide sequence encoding at least one catabolic enzyme of the invention is a chemical system. The chemical systems include, but are not limited to calcium phosphate precipitation, liposomes and polymeric carriers. In some embodiments, the chemical system is based on calcium phosphate precipitation, such as calcium phosphate nano-composite particles encapsulating DNA (see, Nouri et al. Calcium phosphate-mediated gene delivery using simulated body fluid (SBF). Int J Pharm. 2012; 434(1-2): 199-208; Bhakta et al. Magnesium phosphate nanoparticles can be efficiently used in vitro and in vivo as non-viral vectors for targeted gene delivery. J Biomed Nanotechnol. 2009; 5(1): 106-114).

In some embodiments, the chemical system to deliver a nucleotide sequence encoding at least one catabolic enzyme of the invention is based on liposomes. In some embodiments, the liposomes are nano-sized. In some embodiments, liposomes conjugated with polyethylene glycol (PEG) and/or other molecules such as ligands and peptides can be used (see, Yang et al. Cationic nucleolipids as efficient siRNA carriers. Org Biomol Chem. 2011; 1(9): 291-296.).

In some embodiments, the chemical system to deliver a nucleotide sequence encoding at least one catabolic enzyme of the invention is based on polymeric carriers. In some embodiments, the polymeric carriers are conjugated to the gene to be delivered. In some embodiments, the polymeric carriers include, but are not limited to chitosan, polyethylenimine (PEI), polylysine, polyarginine, polyamino ester, Polyamidoamine Dendrimers (PAMAM), Poly (lactide-co-glycolide), and PLL, such as those described in Choi et al., Enhanced transfection efficiency of PAMAM dendrimer by surface modification with 1-arginine. J Control Release. 2004; 3(99): 445-456; Pfeifer et al., Poly(ester-anhydride):poly(beta-amino ester) micro- and nanospheres: DNA encapsulation and cellular transfection. Int J Pharm. 2005; 304(1-2): 210-219; Anderson et al., Structure/property studies of polymeric gene delivery using a library of poly(beta-amino esters). Mol Ther. 2005; 3(11): 426-434; Hwang et al., Effects of structure of beta-cyclodextrin-containing polymers on gene delivery. Bioconjugate Chem. 2001; 2(12): 280-290; Kean et al., Trimethylated chitosans as non-viral gene delivery vectors: cytotoxicity and transfection efficiency. J Control Release. 2005; 3(103): 643-653.

In some embodiments, administration of a catabolic enzyme comprises the administration of at least one catabolic enzyme polypeptide or fragment thereof of the present invention. As used herein, the terms “polypeptide” and “protein” are used interchangeably herein.

The invention also envisions and encompasses the use of functional variants or fragments of the intralysosomal catabolic enzyme described herein. As used herein, the phrase “a biologically active variant” or “functional variant” with respect to a protein refers to an amino acid sequence that is altered by one or more amino acids with respect to a reference sequence, while still maintains substantial biological activity of the reference sequence. The variant can have “conservative” changes, wherein a substituted amino acid has similar structural or chemical properties, e.g., replacement of leucine with isoleucine. The following table shows exemplary conservative amino acid substitutions.

| | | Highly Conserved | Conserved |
|---|---|---|---|
| | Very Highly- | Substitutions | Substitutions |
| Original | Conserved | (from the Blosum90 | (from the Blosum65 |
| Residue | Substitutions | Matrix) | Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, | Arg, Asp, Gln, Glu, His, |
| | | Ser, Thr | Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, His, |
| | | Lys, Met | Lys, Met, Ser |
| Glu | Asp | Asp, Gln, Lys | Arg, Asn, Asp, Gln, His, |
| | | | Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software. For polynucleotides, a variant comprises a polynucleotide having deletions (i.e., truncations) at the 5′ and/or 3′ end; deletion and/or addition of one or more nucleotides at one or more internal sites in the reference polynucleotide; and/or substitution of one or more nucleotides at one or more sites in the reference polynucleotide. As used herein, a “reference” polynucleotide comprises a nucleotide sequence produced by the methods disclosed herein. Variant polynucleotides also include synthetically derived polynucleotides, such as those generated, for example, by using site directed mutagenesis but which still comprise genetic regulatory element activity. Generally, variants of a particular polynucleotide or nucleic acid molecule, or polypeptide of the invention will have at least about 60%, 65%, 70%, 75%, 80%, 85%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or more sequence identity to that particular polynucleotide/polypeptides as determined by sequence alignment programs and parameters as described elsewhere herein.

In some embodiments, a gene that can hybridize with the nucleic acid sequences encoding the catabolic enzymes of the present invention under stringent hybridization conditions can be used. The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na⁺ ion, typically about 0.01 to 1.0 M Na+ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001. In some embodiments, stringent conditions are hybridization in 0.25 M Na₂HPO₄ buffer (pH 7.2) containing 1 mM Na₂EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

The definition of each catabolic enzyme includes sequences having high similarity or identity to the nucleic acid sequences and/or polypeptide sequences of the specific catabolic enzymes mentioned herein. As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

The invention also includes biologically active fragments of the catabolic enzymes described herein. These biologically active fragments may comprise at least 10, 20, 50, 100, 150, 200, 250, 300, 350, 400, 450, or more amino acid residues and retain one or more activities associated with the catabolic enzymes described herein. Such fragments may be obtained by deletion mutation, by recombinant techniques that are routine and well-known in the art, or by enzymatic digestion of the catabolic enzyme(s) of interest using any of a number of well-known proteolytic enzymes. The invention further includes nucleic acid molecules which encode the above described variant enzymes and enzyme fragments.

In some embodiments, the methods comprise administering to the subject a composition comprising a therapeutically effective amount or prophylactically effective amount of at least one catabolic enzyme. The term “therapeutically effective amount” as used herein, refers to the level or amount of one or more catabolic enzymes needed to treat amyloidosis, or reduce or prevent injury or damage, optionally without causing significant negative or adverse side effects. A “prophylactically effective amount” refers to an amount of a catabolic enzyme sufficient to prevent or reduce severity of a future disease or condition associated with amyloidosis when administered to a subject who is susceptible and/or who may develop amyloidosis or a condition associated with amyloidosis.

In some embodiments, instead of or in addition to administering a polynucleotide sequence encoding a catabolic enzyme of the present invention, the methods comprise administering a composition comprising a polypeptide comprising a catabolic enzyme of the present invention or a biologically active fragment thereof directly to the subject in need.

In some embodiments, the catabolic enzyme is targeted to the intralysosomal space. In some embodiments, the catabolic enzyme to be administered comprises one or more signals which help with sorting the polypeptide to lysosome. In some embodiments, the signal can be a lysosomal localization signal polypeptide, a monosaccharide (including derivatives), a polysaccharide, or combinations thereof.

In some embodiments, the signal is mannose-6 phosphate. A catabolic enzyme comprising a mannose-6 phosphate can be targeted to lysosomes with the help of a mannose-6 phosphate receptor.

In some embodiments, the signal is not dependent on mannose-6 phosphate. In some embodiments, the signal is a signal peptide. In some embodiments, the signal peptide is located at the N-terminal, the C-terminal, or elsewhere in the intralysosomal catabolic enzyme to be administered. In some embodiments, the signal peptides include, but are not limited to the DXXLL type (SEQ ID NO: 13), [DE]XXXL[LI] type (SEQ ID NO: 14), and YXXO type (SEQ ID NO: 15). See Bonifacino et al., Signals for sorting of transmembrane proteins to endosomes and lysosomes, Annu. Rev. Biochem. 72 (2003) 395-447; and Brualke et al. (Sorting of lysosomal proteins, Biochimica et Biophysica Acta 1793 (2009) 605-614), each of which is incorporated by reference in its entirety.

In some embodiments, the signal peptides belong to the DXXLL type, such as those identified in MPR300/CI-MPR (SFHDDSDEDLL, SEQ ID NO: 16), MPR46/CD-MPR (EESEERDDHLL, SEQ ID NO: 17), Sortilin (GYHDDSDEDLL, SEQ ID NO: 18), SorLA/SORL1 (ITGFSDDVPMV, SEQ ID NO: 19), GGA1 (1) (ASVSLLDDELM, SEQ ID NO: 20), GGA1 (2) (ASSGLDDLDLL, SEQ ID NO: 21), GGA2 (VQNPSADRNLL, SEQ ID NO: 22), and GGA3 (NALSWLDEELL, SEQ ID NO: 23).

In some embodiments, the signal peptides belong to the [DE]XXXL[LI] type, such as those identified in LIMP-II (DERAPLI, SEQ ID NO: 24), NPC1 (TERERLL, SEQ ID NO: 25), Mucolipin-1 (SETERLL, SEQ ID NO: 26), Sialin (TDRTPLL, SEQ ID NO: 27), GLUT8 (EETQPLL, SEQ ID NO: 28), Invariant chain (Ii) (1) (DDQRDLI, SEQ ID NO: 29), and Invariant chain (Ii) (2) (NEQLPML, SEQ ID NO: 30).

In some embodiments, the signal peptides belong to the YXXO type, such as those identified in LAMP-1 (GYQTI, SEQ ID NO: 31), LAMP-2A (GYEQF, SEQ ID NO: 32), LAMP-2B (GYQTL, SEQ ID NO: 33), LAMP-2C (GYQSV, SEQ ID NO: 34), CD63 (GYEVM, SEQ ID NO: 35), CD68 (AYQAL, SEQ ID NO: 36), Endolyn (NYHTL, SEQ ID NO: 37), DC-LAMP (GYQRI, SEQ ID NO: 38), Cystinosin (GYDQL, SEQ ID NO: 39), Sugar phosphate exchanger 2 (GYKEI, SEQ ID NO: 40), and acid phosphatase (GYRHV, SEQ ID NO: 41).

In some embodiments, the catabolic enzyme is targeted to remain outside the cell, i.e., the enzyme is modified to act extracellularly. In some embodiments, the catabolic enzyme to be administered lacks one or more signals that would otherwise target the polypeptide to the lysosome. In some embodiments, the catabolic enzyme lacks one or more mannose-6 phosphate (i.e., M6P) signals, thereby precluding entry of the catabolic enzyme into the cell. In some embodiments, the catabolic enzyme is recombinantly engineered to lack one or more mannose-6 phosphate signal. Not bound by any theory, it is generally understood in the art that reduced M6P content lowers the binding affinity of a recombinant enzyme for M6P receptors and decreases its cellular uptake and thereby allows the enzyme to remain outside the cell.

Methods for reducing the M6P content of a recombinant protein, e.g., a catabolic enzyme, are known in the art. See, e.g., U.S. Pat. No. 8,354,105, which is herein incorporated by reference in its entirety. In some embodiments, the mannose content of a recombinant catabolic enzyme may be reduced by manipulating the cell culture conditions such that the glycoprotein produced by the cell has low-mannose content. As used herein, the term “low-mannose content” refers to catabolic enzyme composition wherein less than about 20%, less than about 15%, less than about 100, less than about 8%, less than about 5%, less than about 4%, less than about 3%, less than about 2%, less than about 1%, or any values between any of these preceding ranges, or even at 0% of the enzymes in the composition have more than 4 mannose residues (i.e., are species of M5 or greater).

In some embodiments, the present invention provides a composition comprising at least two catabolic enzymes, wherein the composition comprises at least one catabolic enzyme that is targeted to the cell lysosome and at least one catabolic enzyme that remains outside the cell. In some embodiments, the catabolic enzymes are selected from protective protein/cathepsin A (PPCA), neuraminidase 1 (NEU1), tripeptidyl peptidase 1 (TPP1), cathepsin B, cathepsin D, cathepsin E, cathepsin K, and cathepsin L. In an exemplary embodiment, the present invention provides a composition comprising at least two catabolic enzymes, wherein the composition comprises a PPCA catabolic enzyme that is targeted to the cell lysosome and a PPCA catabolic enzyme that remains outside the cell. In some embodiments, the ratio of the intralysosomal catabolic enzyme to the extracellular catabolic enzyme on a percentage basis within the composition is at least 5%:95%. In further embodiments, the ratio of the intralysosomal catabolic enzyme to the extracellular catabolic enzyme on a percentage basis within the composition is at least 10%:90%, at least 15%:85%, at least 20%:80%, at least 25%:75%, at least 30%:70%, at least 35%:65%, at least 40%:60%, at least 45%:55%, at least 50%:50%, at least 55%:45%, at least 60%:40%, at least 65%:35%, at least 70%:30%, at least 75%:25%, at least 80%:20%, at least 85%:15%, at least 90%:10%, or at least 95%:5%.

In some embodiments, the methods of the present invention comprise administering to the subject a composition comprising a therapeutically effective amount of at least two, three, or more catabolic enzymes. In some embodiments, the methods comprise increasing the expression, activity, and/or concentration of at least two, three, or more catabolic enzymes in the subject. In some embodiments, the methods comprise administering to the subject a composition comprising an expression cassette comprising one or more polynucleotide sequences encoding at least two, three, or more catabolic enzymes. In some embodiments, the methods comprise administering to the subject one or more expression cassettes comprising at least two, three or more polynucleotide sequences encoding at least two, three or more catabolic enzymes. In some embodiments, the methods comprise administering to the subject a therapeutically effective amount of a first catabolic enzyme, and an expression cassette comprising a polynucleotide sequence encoding a second catabolic enzyme. In some embodiments, two or more catabolic enzymes are selected from the group consisting of protective protein/cathepsin A (PPCA), neuraminidase 1 (NEU1), tripeptidyl peptidase 1 (TPP1), cathepsin B, cathepsin D, cathepsin E, cathepsin K, and cathepsin L. In some embodiments, at least two catabolic enzymes are PPCA and NEU1.

In some embodiments, administration of the at least one catabolic enzyme is employed to prevent the formation of amyloid. In other embodiments, administration of the at least one catabolic enzyme is employed to degrade amyloid that has already formed. In some embodiments, administration of the at least one catabolic enzyme is employed to prevent the formation of one or more amyloid oligomers. In some embodiments, administration of the at least one catabolic enzyme is employed to prevent the formation of one or more amyloid fibrils. In some embodiments, administration of the at least one catabolic enzyme is employed to degrade one or more amyloid oligomers after it has already formed. In some embodiments, administration of the at least one catabolic enzyme is employed to degrade one or more amyloid fibrils after it has already formed.

In some embodiments, the methods of the present invention provided herein further comprise administering a composition (e.g. a pharmaceutical composition) comprising at least one catabolic enzyme or fragment thereof with at least one additional drug for treating or preventing amyloidosis.

In some embodiments, the at least one additional drug is a steroid. In some embodiments, the steroid is dexamethasone, cortisone, hydrocortisone, methylprednisolone, prednisolone, prednisone, triamcinolone or any combination thereof.

In some embodiments, the at least one additional drug is a non-steroid agent. In some embodiments, such non-steroid agent is diclofenac, flufenamic acid, flurbiprofen, diflunisal, detoprofen, diclofenac, etodolac, fenoprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate or any combination thereof.

In some embodiments, the at least one additional drug is a chemotherapy agent. In some embodiments, the chemotherapy agent is selected from the group consisting of cyclophosphamide (e.g., Cytoxan, Neosar) and melphalan (e.g., Alkeran).

In some embodiments, at least one additional drug is an anti-inflammatory medication, when the subject has inflammatory symptoms.

In some embodiments, the at least one additional drug is an antibiotic, when the subject has infection symptoms. In some embodiments, the infection is a chromic infection. In some embodiments, the infection is a microbial infection.

In some embodiments, the at least one additional drug is a Carbonic Anhydrase (CA) enzyme (e.g., CA-I, CA-II, CA-III, CA-IV, CA-V, CA-VI, and CA-VII) and/or agents that can increase the activity of a Carbonic Anhydrase enzyme in the subject.

In some embodiments, at least one additional drug is a disease modifying antirheumatic drug (DMARD). In some embodiments, the DMARD is cyclosporine, azathioprine, methotrexate, leflunomide, cyclophosphamide, hydroxychloroquine, sulfasalazine, D-penicillamine, minocycline, gold, or any combination thereof.

In some embodiments, the at least one additional drug is a recombinant protein. In some embodiments, the recombinant protein is ENBREL® (etanercept, a soluble TNF receptor) or REMICADE® (infliximab, a chimeric monoclonal anti-TNF antibody).

In some embodiments, the one or more additional drugs is/are selected from melphalan, dexamethasone, bortezomib, lenalidomide, vincristine, doxorubicin, cyclophosphamide and pomalidomide.

In some embodiments, the methods of the present invention further comprise the administration of one or more drugs that acidifies the lysosome. As used herein, drugs that acidify the lysosome are drugs capable of lowering the lysosomal pH of a target cell. Accordingly, in some embodiments, the present invention provides a method of treating or preventing amyloidosis in a subject comprising administering to the subject a composition comprising a therapeutically effective amount of at least one catabolic enzyme or a biologically active fragment thereof, wherein the subject is also administered one or more drugs that acidifies the lysosome. As described herein, when performing a combination therapy, the two or more drugs (e.g., a catabolic enzyme or a biologically active fragment thereof and a drug that acidifies the lysosome) can be administered simultaneously or sequentially in any order.

In some embodiments, the drug that acidifies the lysosome is selected from an acidic nanoparticle, a catecholamine, a β-adrenergic receptor agonist, an adenosine receptor agonist, a dopamine receptor agonist, an activator of the cystic fibrosis transmembrane conductance regulator (CFTR), cyclic adenosine monophosphate (cAMP), a cAMP analog, and an inhibitor of glycogen synthase kinase-3 (GSK-3).

In some embodiments, the drug that acidifies the lysosome is an acidic nanoparticle. Acidic nanoparticles have been shown to localize to lysosomes and reduce lysosomal pH. See Baltazar et al., 2012, PloS ONE 7(12): e49635 and Lee et al., 2015, Cell Rep. 12(9): 1430-44, both of which are herein incorporated by reference in their entireties. In some embodiments, the acidic nanoparticle is a polymeric acidic nanoparticle. In some embodiments, the polymeric acidic nanoparticle is a poly (DL-lactide-co-glycolide) (PLGA) acidic nanoparticle. In a specific embodiment, the PLGA acidic nanoparticle comprises PLGA Resomer RG 503 H. In some embodiments, the PLGA acidic nanoparticle comprises PLGA Resomer RG 502 H. In other embodiments, the polymeric acidic nanoparticle is a poly (DL-lactide) (PLA) acidic nanoparticle. In a specific embodiment, the PLA acidic nanoparticle comprises PLA Resomer R 203 S. In some embodiments, the acid number of the acidic nanoparticle is between about 0.5 mg KOH/g to about 8 mg KOH/g. In some embodiments, the acid number of the acidic nanoparticle is between about 1 mg KOH/g to about 6 mg KOH/g. In some embodiments, the acid number of the acidic nanoparticle is selected from about 1 mg KOH/g, about 2 mg KOH/g, about 3 mg KOH/g, about 4 mg KOH/g, about 5 mg KOH/g, or about 6 mg KOH/g. In a specific embodiment, the acid number of the acidic nanoparticle is about 3 mg KOH/g. In some embodiments, the nanoparticle size is about 50 nm to about 800 nm. In some embodiments, the nanoparticle size is about 100 nm to about 600 nm. In a specific embodiment, the nanoparticle size is about 350 nm to about 550 nm. In a further specific embodiment, the nanoparticle size is about 375 nm to about 400 nm. In an exemplary embodiment, the acidic nanoparticle is spherical. In some embodiments, the nanoparticles are targeting a specific transport process in the brain, which enhance drug transport through the blood-brain barrier (BBB). In some embodiments, such transport processes include, but are not limited to: (1) nanoparticles open TJs between endothelial cells or induce local toxic effect which leads to a localized permeabilization of the BBB allowing the penetration of the drug in a free form or conjugated with the nanoparticles; (2) nanoparticles pass through endothelial cell by transcytosis; (3) nanoparticles are transported through endothelial cells by endocytosis, where the content is released into the cell cytoplasm and then exocytosed in the endothelium abluminal side; and (4) a combination of several of the mechanisms. In some embodiments, the receptors targeted by nanoparticles are transferrin and low-density lipo-protein receptors. In some embodiments, the targeting can be achieved by peptides, proteins, or antibodies, which can be physically and/or chemically immobilized on the nanoparticles. In some embodiments, the nanoparticles are coated with one or more apolipoproteins, such as apolipoprotein AII, B, CII, E, and/or J (see, Kreuter et al., (2002, DOI: 10.1080/10611860290031877). For more nanoparticle-mediated brain drug delivery compositions and methods, see Saraiva et al. (Journal of Controlled Release, 2016, 235:34-37). Each of the references mentioned herein is incorporated by reference in its entirety.

In some embodiments, the drug that acidifies the lysosome is a catecholamine. Catecholamines have been shown to reduce lysosomal pH. See Liu et al., 2008, Invest Ophthalmol Vis Sci. 49(2): 772-780, which is herein incorporated by reference in its entirety. In some embodiments, the catecholamine is selected from epinephrine, metanephrine, synephrine, norepinephrine, normetanephrine, octopamine or norphenephrine, dopamine, and dopa. In exemplary embodiment, the catecholamine is selected from epinephrine, norepinephrine, and dopamine.

In some embodiments, the drug that acidifies the lysosome is a β-adrenergic receptor agonist. β-adrenergic receptor agonists have been shown to reduce lysosomal pH. See Liu et al., 2008, Invest Ophthalmol Vis Sci. 49(2): 772-780. Examples of β-adrenergic receptor agonists may be found in US Patent Publication No. 2012/0329879, which is herein incorporated by reference in its entirety. In some embodiments, the β-adrenergic receptor agonist is selected from isoproterenol, metaproterenol, formoterol, salmeterol, salbutamol, albuterol, terbutaline, fenoterol, and vilanterol. In an exemplary embodiment, the β-adrenergic receptor agonist is isoproterenol.

In some embodiments, the drug that acidifies the lysosome is an adenosine receptor agonist. Adenosine receptor agonists have been shown to reduce lysosomal pH. See Liu et al., 2008, Invest Ophthalmol Vis Sci. 49(2): 772-780. In an exemplary embodiment, the adenosine receptor agonist is a non-specific adenosine receptor agonist or an A_{2A} adenosine receptor agonist. Examples of A_{2A} adenosine receptor agonists may be found in US Patent Publication No. 2012/0130481, which is herein incorporated by reference in its entirety. In some embodiments, the adenosine receptor agonist is selected from 5′-N-ethylcarboxamidoadenosine (NECA), CGS21680, 2-phenylaminoadenosine, 2-[para-(2carboxyethyl)phenyl]amino-5′N-ethylcarboxamidoadenosine, SRA-082, 5′-N-cyclopropylcarboxamidoadenosine, 5′N-methylcarboxamidoadenosine and PD-125944.

In some embodiments, the drug that acidifies the lysosome is a dopamine receptor agonist. Dopamine receptor agonists have been shown to reduce lysosomal pH. See Guha et al., 2014, Adv Exp MedBiol. 801: 105-111, which is herein incorporated by reference in its entirety. In some embodiments, the dopamine receptor agonist is selected from A68930, A77636, A86929, SKF81297, SKF82958, SKF38393, SKF89145, SKF89626, dihydrexidine, dinapsoline, dinoxyline, doxanthrine, fenoldopam, 6-Br-APB, stepholidine, CY-208243, 7,8-Dihydroxy-5-phenyl-octahydrobenzo[h]isoquinoline, cabergoline, and pergolide. In an exemplary embodiment, the dopamine receptor agonist is selected from A68930, A77636, and SKF81297. In a further exemplary embodiment, the dopamine receptor agonist is SKF81297, also known as 6-chloro-1-phenyl-2,3,4,5-tetrahydro-1H-3-benzazepine-7,8-diol.

In some embodiments, the drug that acidifies the lysosome is an activator of the cystic fibrosis transmembrane conductance regulator (CFTR). Activators of CFTR have been shown to reduce lysosomal pH. See Liu et al., 2012, Am J Physiol Cell Physiol 303: C160-9, which is herein incorporated by reference in its entirety. In some embodiments, the CFTR activator is selected from CFTR_{Act}01 to CFTR_{Act}17. See Ma et al., J Biol Chem 277: 37235-37241. In an exemplary embodiment, the CFTR activator is selected from CFTR_{Act}11 and CFTR_{Act}16, having the following structures:

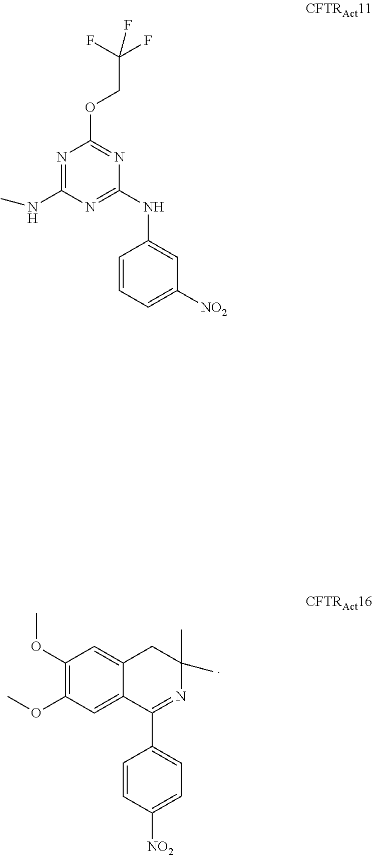

In some embodiments, the CFTR activator is co-administered with forskolin.

In some embodiments, the drug that acidifies the lysosome is cAMP or a cAMP analog. cAMP and/or cAMP analogs have been shown to reduce lysosomal pH. See Liu et al., 2008, Invest Ophthalmol Vis Sci. 49(2): 772-780. For instance, the cell-permeable analogs chlorophenylthio-cAMP (cpt-cAMP) and 8-bromo-cAMP have the ability to lower lysosomal pH in cells. In some embodiments, cAMP and/or a cAMP analog may be administered in a cocktail comprising 3-isobutyl-1-methylxanthine (IBMX) and forskolin. For example, in one embodiment, a cocktail comprising IBMX, forskolin, and cpt-cAMP may be administered to acidify the lysosome. In some embodiments, the cAMP analog is selected from 9-pCPT-2-O-Me-cAMP, Rp-cAMPS, 8-Cl-cAMP, Dibutyryl cAMP, pCPT-cAMP, N6-monobutyryladenosine 3′,5′-cyclic monophosphate, and PDE inhibitors.

In some embodiments, the drug that acidifies the lysosome is an inhibitor of glycogen synthase kinase-3 (GSK-3). GSK-3 inhibitors have been shown to be effective in reducing the lysosomal pH. See Avrahami et al., 2013, Commun Integr Biol 6(5): e25179, which is herein incorporated by reference in its entirety. For instance, the competitive GSK-3 inhibitor, L803-mts, has been shown to facilitate acidification of the lysosome by inhibiting GSK-3 activity, which acts to impair lysosomal acidification. Accordingly, in one embodiment, the inhibitor of GSK-3 is the cell permeable peptide, L803-mts (SEQ ID NO: 72). Suitable GSK-3 inhibitors may be found in US Patent Publication Nos. 2013/0303441 and 2015/0004255, which are herein incorporated by reference in their entireties. In some embodiments, the GSK-3 inhibitor is selected from 2′Z,3′E)-6-bromoindirubin-3′-acetoxime, TDZD-8 (4-Benzyl-2-methyl-1,2,4-thiadiazolidine-3,5-dione), S13216763 (3-(2,4-Dichlorophenyl)-4-(1-methyl-1H-indol-3-yl), NP-103. 2-Thio(3-iodobenzyl)-5-(1-pyridyl)-[1,3,4]-oxadiazole, L803, L803-mts, and GF-109203X (2-[1-(3-Dimethylaminopropyl)indol-3-yl]-3-(indol-3-yl)maleimide) and pharmaceutically acceptable salts and mixtures thereof.

In some embodiments, the methods of the present invention further comprise the administration of one or more drugs that promotes autophagy. As used herein, drugs that promote autophagy can promote the intracellular degradation system that delivers cytoplasmic constituents to the lysosome. Accordingly, in some embodiments, the present invention provides a method of treating or preventing amyloidosis in a subject comprising administering to the subject a composition comprising a therapeutically effective amount of at least one catabolic enzyme or a biologically active fragment thereof, and one or more drugs that promotes autophagy. In some embodiments, the present invention provides a method of treating or preventing amyloidosis in a subject comprising administering to the subject a composition comprising a therapeutically effective amount of at least one catabolic enzyme or a biologically active fragment thereof, wherein the subject is also administered one or more drugs that acidifies the lysosome and/or endosome, and one or more drugs that promotes autophagy. In some embodiments, the drug that acidifies the lysosome and/or endosome, and the drug that promotes autophagy can be the same drug, or different drugs. As described herein, when performing a combination therapy, the drugs (e.g., a catabolic enzyme or a biologically active fragment thereof, a drug that acidifies the lysosome and/or endosome, and/or a drug that promotes autophagy) can be administered simultaneously or sequentially in any order. Without wishing to be bound by any particular theory, a treatment of therapeutic catabolic enzyme or a biologically active fragment thereof with an agent that can cause lysosome and/or endosome acidification and/or an agent that can promote autophagy is capable of lowering pH to optimal conditions for enzymatic proteolysis, and improving lysosomal proteolysis power.

In some embodiments, autophagy promoting reagents include, but are not limited to reagents that directly or indirectly promote autophagy such as TFEB activators, PPAR agonists, PGC-1α activators, LSD1 inhibitors, mTOR inhibitors, GSK3 inhibitors, etc.

In some embodiments, the drug promotes autophagy via activation of Transcription factor EB (TFEB) pathway. TFEB is a master gene for lysosomal biogenesis. It encodes a transcription factor that coordinates expression of lysosomal hydrolases, membrane proteins and genes involved in autophagy. TFEB overexpression in cultured cells induced lysosomal biogenesis and increased the degradation of complex molecules. TFEB is activated by PGC-1α and promotes reduction of htt aggregation and neurotoxicity.

In some embodiments, the drug that promotes autophagy via activation of TFEB pathway is an activator of TFEB. In some embodiments, such TFEB activator include, but are not limited to C1 (Song et al, 2016, Autophagy, 12(8):1372-1389), and 2-hydroxypropyl-3-cyclodextrin (Kilpatrick et al., 2015, PLOS ONE DOI:10.1371/journal.pone.0120819). Each of the references mentioned herein is incorporated by reference in its entirety.

In some embodiments, the drug that promotes autophagy via activation of TFEB pathway is an agent that can activate peroxisome proliferator-activated receptor gamma coactivator 1-α (PGC-1α). In some embodiments, such activators of PGC-1α include, but are not limited to, pyrroloquinoline quinone, resveratrol, R-α-lipoic acid (ALA), ALA/acetyl-L-carnitine (ALC), flavonoids, isoflavones and derivatives (e.g., quercetin, daidzein, genistein, biochanin A, and formononetin). See, Das and Sharma 2015 (CNS & Neurological Disorders—Drug Targets, 2015, 14, 1024-1030.) Each of the references mentioned herein is incorporated by reference in its entirety.

In some embodiments, the drug promotes autophagy via activation of peroxisome proliferator-activated receptor gamma coactivator 1-α (PGC-1α) and/or Forehead box 03 (FOXO3). PGC-1α is a master regulator of mitochondrial biogenesis. PGC-1α interacts with the nuclear receptor PPAR-γ, which permits the interaction of this protein with multiple transcription factors. This protein can interact with, and regulate the activities of, cAMP response element-binding protein (CREB) and nuclear respiratory factors (NRFs). It provides a direct link between external physiological stimuli and the regulation of mitochondrial biogenesis, and is a major factor that regulates muscle fiber type determination. FOXO3 is a transcription factor that can be inhibited and translocated out of the nucleus on phosphorylation by protein such as Akt/PKB in the PI3K signaling pathway.

In some embodiments, a drug that promotes autophagy via PGC-1α and/or FOXO3 activation is an inhibitor of Lysine (K)-specific demethylase 1A (LSD1). LSD1 is a flavin-dependent monoamine oxidase, which can demethylate mono- and bi-methylated lysines. LSD1 has roles critical in embryogenesis and tissue-specific differentiation. In some embodiments, such LSD1 inhibitors include, but are not limited to, 1-(4-methyl-1-piperazinyl)-2-[[(1R*,2S*)-2-[4-phenylmethoxy)phenyl]cyclopropyl]amino]ethanone dihydrochloride (RN-1; Cui et al., 2015, Blood 2015 126:386-396), CBB1001-1009 (Wang et al., 2011, Cancer Res. 2011 Dec. 1; 71(23): 7238-7249), TCP, Pargyline, CGC-11047, and Namolone (Pieroni et al., 2015, European Journal of Medicinal Chemistry 92 (2015) 377e386), phenelzine analogues (Prusevich et al., ACS Chem. Biol. 2014, 9, 1284-1293), and those described in WO2015156417, which is herein incorporated by reference in its entirety. In some embodiments, one or more LSD1 inhibitors are used. In some embodiments, both RN-1 and a LSD1 inhibitor described in WO2015156417 are used. WO2015156417 describes inhibitors of LSD1 represented by formula I:

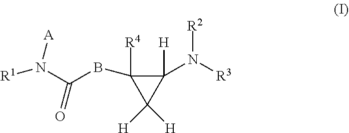

wherein, A is an optionally substituted heterocyclic group. or an optionally substituted hydrocarbon group; B is a ring selected from
(1) a 5- or 6-membered aromatic heterocycle optionally fused with an optionally substituted 5- or 6-membered ring, and
(2) a benzene ring fused with an optionally substituted 5- or 6-membered ring, wherein the ring represented by B is optionally substituted, and binds, via two adjacent carbon atoms with one atom in between, to a group represented by the formula

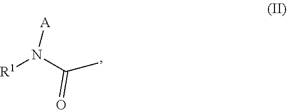

and a group represented by the formula

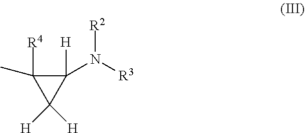

R¹, R², R³ and R⁴ are each independently a hydrogen atom, an optionally substituted hydrocarbon group or an optionally substituted heterocyclic group;
A and R¹ are optionally bonded with each other to form, together with the adjacent nitrogen atom, an optionally substituted cyclic group; and
R² and R³ are optionally bonded with each other to form, together with the adjacent nitrogen atom, an optionally substituted cyclic group, or a salt thereof. Such LSD1 inhibitors are more specific with less side effect and good blood-brain barrier penetration.

In some embodiments, the LSD1 inhibitors are selected from the group consisting of the following compounds (compounds 1-30), and salts, stereoisomers, geometric isomers, tautomers, oxynitrides, enantiomers, diastereoisomers, racemates, prodrugs, solvates, metabolites, esters, and mixtures thereof:

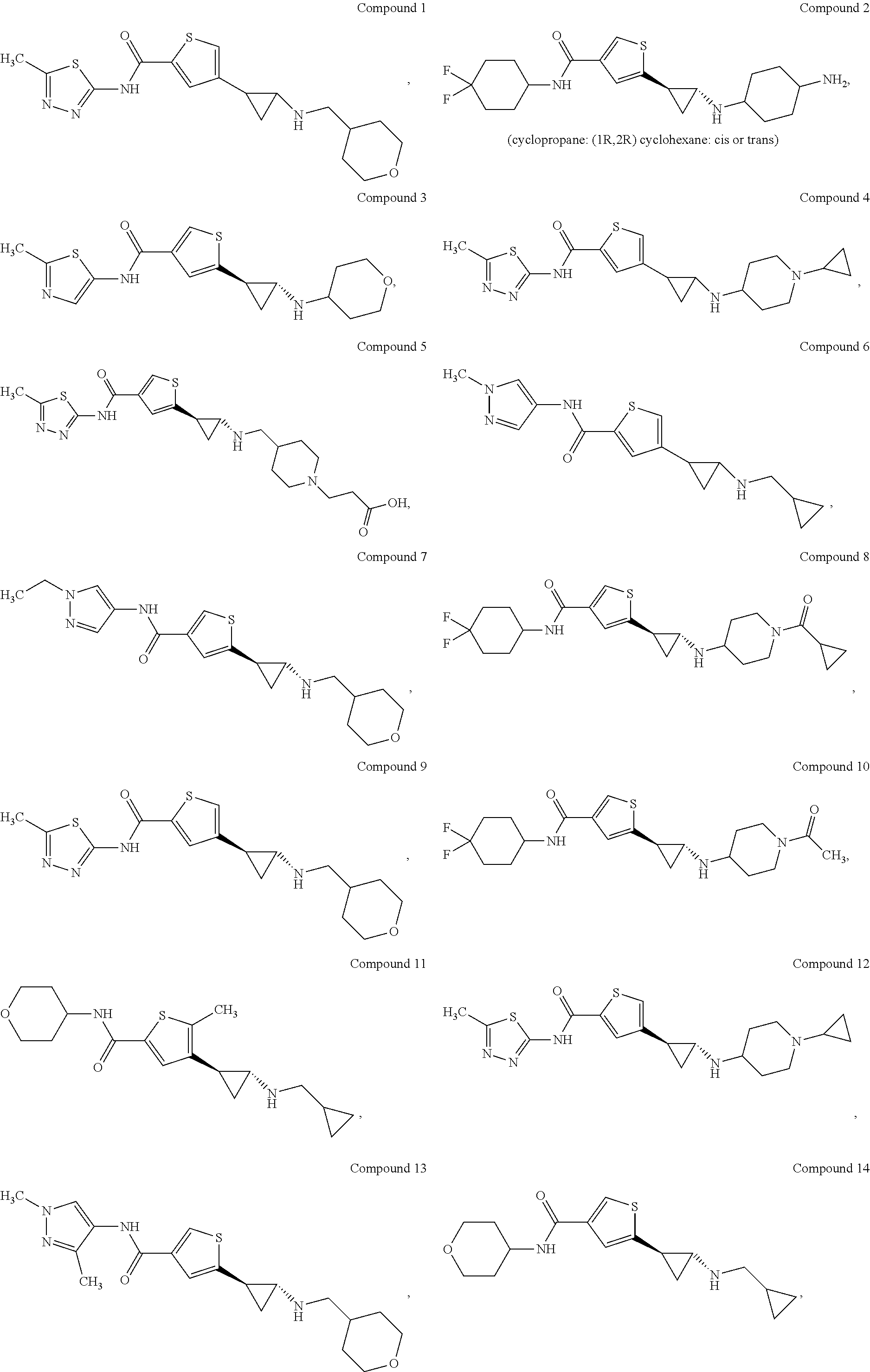

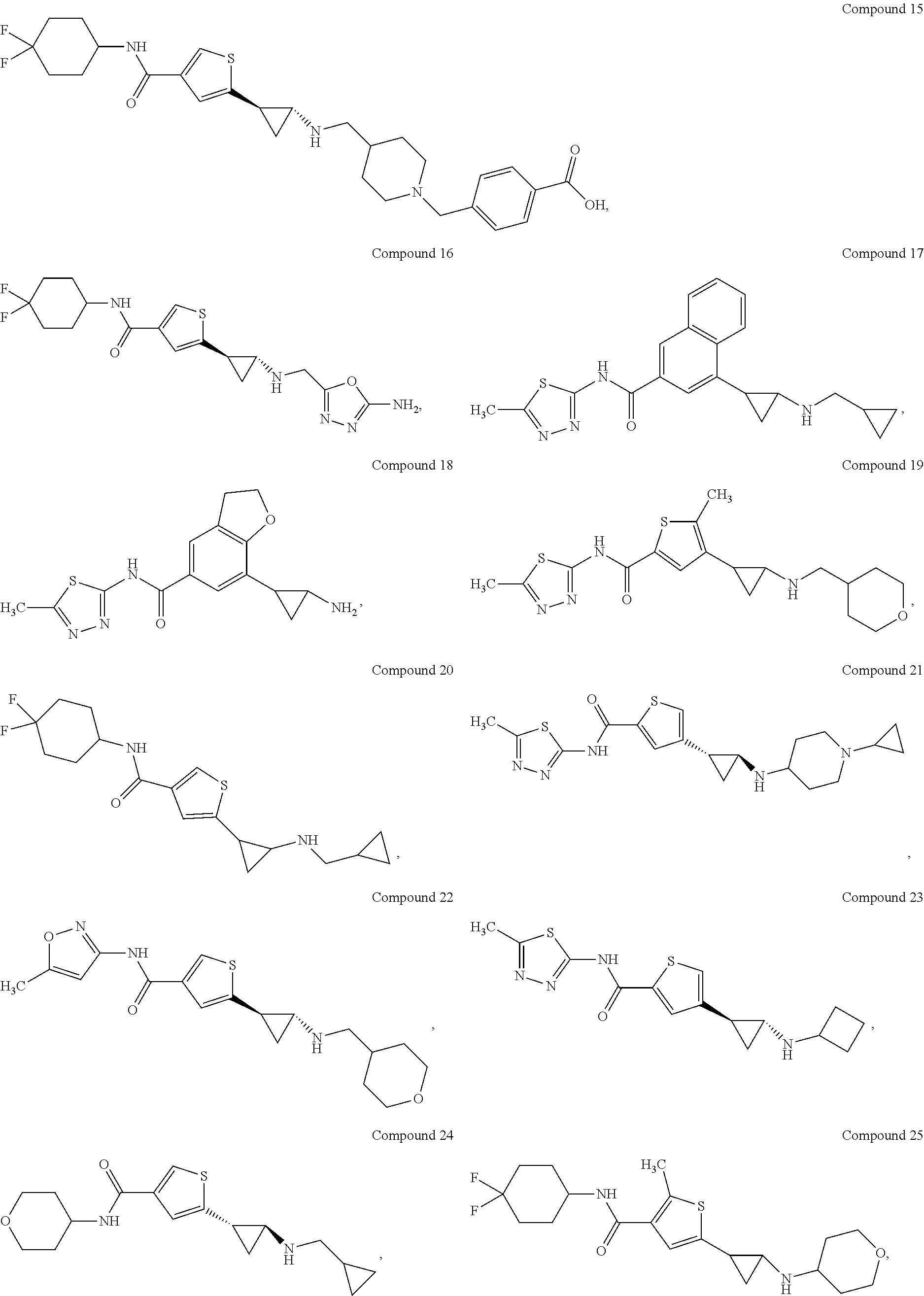

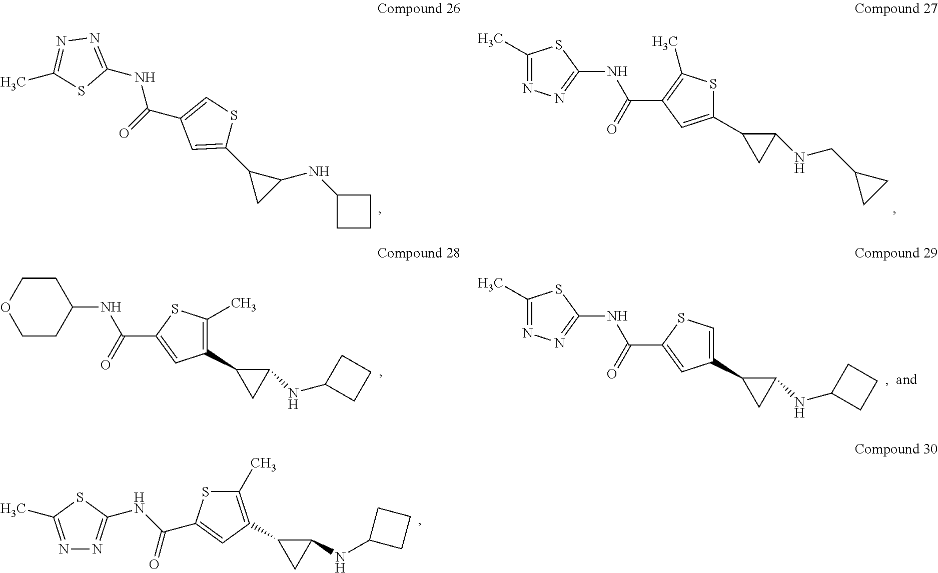 [SMILES: Cc1nnc(NC(=O)c2csc(C3CC3NC3CCC3)c2)s1 ; Cc1nnc(NC(=O)c2cc(C3CC3NCC3CC3)sc2C)s1 ; Cc1sc(C(=O)NC2CCOCC2)cc1[C@@H]1C[C@H]1NC1CCC1 ; Cc1nnc(NC(=O)c2cc([C@@H]3C[C@H]3NC3CCC3)cs2)s1 ; Cc1nnc(NC(=O)c2cc([C@H]3C[C@@H]3NC3CCC3)c(C)s2)s1]

In one embodiment, the LSD1 inhibitor to be co-administered with a catabolic enzyme of the present invention or a biologically active fragment thereof is compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or any mixtures thereof.

In some embodiments, the drug is capable of modify the activity of a regulator or a co-activator of PGC-1α. Such regulators or co-activators of PGC-1α include, but are not limited to, Parkin Interacting Substrate (PARIS), Sirtuin 1 (SIRTI), 5′ AMP-activated protein kinase(AMPK), General control of amino acid synthesis protein 5 (GCN5), Nuclear respiratory factor 1, 2(NRF-1,2), Glycogen synthase kinase 3β (GSK3β), Peroxisome proliferator-activated receptor-α,β/δ,γ (PPAR-α,β/δ,γ), p38 mitogen-activated protein kinase (p38MAPK), Estrogen-related receptors (ERRs), myocyte enhancer factor-2 (MEF2), and Thyroid hormone receptor (TR), see Das and Sharma (CNS & Neurological Disorders—Drug Targets, 2015, 14, 1024-1030). Each of the references mentioned herein is incorporated by reference in its entirety.

In some embodiments, the drug that promotes autophagy is a Peroxisome proliferator-activated receptor (PPAR) agonist. PPARs are nuclear receptor proteins that function as transcription factors regulating the expression of genes. They are critical in the regulation of cellular differentiation, development, and metabolism and tumorigenesis.

In some embodiments, the PPAR is selected from PPARα, PPARβ/δ, and PPARγ. In some embodiments, the PPAR agonist is a PPARα agonist, including but not limited to amphipathic carboxylic acids (e.g., clofibrate, gemfibrozil, ciprofibrate, bezafibrate, and fenofibrate), fibrate, ureidofibrate, oxybenzylglycine, triazolone, agonists containing a 2,4-dihydo-3H-1,2,4 triazole-3-one (triazolone) core (e.g., LY518674), BMS-687453, Wy-14643, GW2331, GW 95798, LY518674, and GW590735.

In some embodiments, the PPAR agonist is a PPARβ/δ agonist, including but not limited to GW501516 (Brunmair; et al. Diabetologia. 49 (11): 2713-22), L-165041, compound 7 (Burdick et al., Cell Signal 2006, 18 (1), 9-20), thiazole, bisaryl substituted thiazoles, non-TZD compounds (e.g., L-165041), L-165041, compound 7 (Burdick et al., Cell Signal 2006, 18 (1), 9-20), 38c (Johnson et al., J Steroid Biochem Mol Biol 1997, 63 (1-3), 1-8), and oxazoles. Each of the references mentioned herein is incorporated by reference in its entirety.

In some embodiments, the PPAR agonist is a PPARγ agonist, including but not limited to thiazolidinediones (TZDs or glitazones), glitazar, indenone, NSAIDs, dihydrocinnamate, β-carboxyethyl rhodamine, and those described in Corona and Duchen, 2016 (Free Radical Biology and Medicine, published online Jun. 23, 2016). In some embodiments, the PPARγ agonist is an endogenous or natural agonist. In some embodiments, the PPARγ agonist is a synthetic agonist. In some embodiments, the PPARγ agonist is selected from the group consisting of eicosanoids prostaglandin-A1, cyclopentenone prostaglandin 15-deoxy-Δ^{12, 14}-Prostaglandin J2 (15D-PGJ2), unsaturated fatty acids such as linoleic acid and socosahexaenoic acid, nitroalkenes such as nitrated oleic acid and linoleic acid, oxidized phospholipids such as hexadecyl azelaoyl phosphatidylcholine and lysophosphatidic acid, non-steroidal anti-inflammatory drugs, such as flufenamic acid, ibuprofen, fenoprofen, and indomethacin, pioglitazone, GW0072, ciglitazone, troglitazone, rosiglitazone, isoglitazone, NC-2100 (Loiodice et al., Curr. Top. Med. Chem. 2011, 11(7):819-39), SB-236636, tesaglitazar, farglitazar, GW1929, compound 14c (Haigh et al., Bioorg Med Chem 1999, 7(5):821-30), SP1818, ragaglitazar, metaglidasen, balaglitazone, and INT131. Each of the references mentioned herein is incorporated by reference in its entirety.

In some embodiments, the PPAR agonist binds to PPARα, PPARβ/δ, and PPARγ, such as bezafibrate, LY465608, indeglitazar, TIPP-204, GW693085, TIPP-401, and TIPP-703. In some embodiments, the PPAR agonist binds to PPARα and PPARγ, such as farglitazar, muraglitazar, tesaglitazar, GW409544, aleglitazar, MK-767, TAK-559, compound 18 (Kojo et al., J. Pharmacol Sci 2003, 93 (3), 347-55), compounds 68, 70, 72, 76 (Felts et al., J Med Chem 2008, 51 (16), 4911-9), metaglidasen, and S-2/S-4 (Suh et al., J Med Chem 2008, 51 (20), 6318-33.). In some embodiments, the PPAR agonist binds to PPARβ and PPARγ, such as compound 23 (Martin et al., J Med Chem 2009, 52(21), 6835-50). More PPARs agonists are described in Nevin et al., 2011 (Current Medicinal Chemistry, 2011, 18, 5598-5623). Each of the references mentioned herein is incorporated by reference in its entirety.

In some embodiments, the drug that promotes autophagy is an inhibitor of mechanistic target of rapamycin (mTOR). mTOR is a serine/threonine-specific protein kinase that belongs to the family of phosphatidylinositol-3 kinase (PI3K) related kinases (PIKKs), see Maiese et al. (Br J Clin Pharmacol, 82(5): 1245-1266), which is herein incorporated by reference in its entirety. mTOR integrates the input from upstream pathways, including insulin, growth factors (such as IGF-1 and IGF-2), and amino acids, and also senses cellular nutrient, oxygen, and energy levels. In some embodiments, mTOR inhibitors include, but are not limited to, an antibody of mTOR, rapamycin and its analogs (e.g., temsirolimus (CCI-779), everolimus (RAD001), ridaforolimus (AP-23573), sirolimus, deforolimus), curcumin (Zhang et al., 2016, Oncotarget), curcumin analogs (Song et al. 2016, Autophagy, 12(8):1372-1389), ATP-competitive mTOR kinase inhibitors, mTOR/PI3K dual inhibitors (dactolisib, BGT226, SF 1126, PKI-587 etc.), deptor (Maiese, Neural Regeneration Research. 2016; 11(3):372-385), and mTORC1/mTORC2 dual inhibitors (TORCdIs, such as sapanisertib (a.k.a. INK128), AZD8055, and AZD2014). Each of the references mentioned herein is incorporated by reference in its entirety.

In some embodiments, the drug that promotes autophagy is an inhibitor of Glycogen synthase kinase 3 (GSK3). GSK3 is a serine/threonine protein kinase that mediates the addition of phosphate molecules onto serine and threonine amino acid residues. In some embodiments, the GSK3 inhibitor is ATP-competitive. In some embodiments, the GSK3 inhibitor is non-ATP competitive. In some embodiments, GSK3 inhibitors include, but are not limited to, an antibody of GSK3, metal cations (e.g., beryllium, copper, lithium, mercury, and tungsten), marine organism-derived drugs (e.g., 6-BIO, dibromocantharelline, hymenialdesine, indirubins, meridianins, manzamine A, palinurine, tricantine), aminopyrimidines (e.g., CT98014, CT98023, CT99021, and TWS119), ketamine, arylindolemaleimide (e.g., SB-216763 and SB-41528), thiazoles (e.g., AR-A014418 and AZD-1080), paullones (e.g., Alsterpaullone, Cazpaullone, Kenpaullone), thiadiazolidindiones (e.g., TDZD-8, NP00111, NP031115, and tideglusib), halomethylketones (e.g., HMK-32), certain peptides (L803-mts), SB415286, SB216763, and CT99021 (Stretton et al., 2015, Biochem. J. (2015) 470, 207-221; Marchand et al., 2015, The Journal of Biological Chemistry, 290(9):5592-5605). Each of the references mentioned herein is incorporated by reference in its entirety.

In some embodiments, the methods of the present invention further comprise the administration of one or more drugs that modulates the lysosome. In some embodiments, drugs that modulate the lysosome may be capable of decreasing the level of Rab5a, a marker of early endosomes. Accordingly, in some embodiments, the present invention provides a method of treating or preventing amyloidosis in a subject comprising administering to the subject a composition comprising a therapeutically effective amount of at least one catabolic enzyme or a biologically active fragment thereof, wherein the subject is also administered one or more drugs that modulates the lysosome. As described herein, when performing a combination therapy, the two or more drugs (e.g., a catabolic enzyme or a biologically active fragment thereof and a drug that modulates the lysosome) can be administered simultaneously or sequentially in any order

In some embodiments, the drug that modulates the lysosome is Z-phenylalanyl-alanyl-diazomethylketone (PADK) or a PADK analog, or a pharmaceutically acceptable salt or ester thereof. In some embodiments, the PADK analog is selected from Z-L-phenylalanyl-D-alanyl-diazomethylketone (PdADK), Z-D-phenylalanyl-L-alanyl-diazomethylketone (dPADK), and Z-D-phenylalanyl-D-alanyl-diazomethylketone (dPdADK). In some embodiments, the drug that modulates the lysosome is Z-phenylalanyl-phenylalanyl-diazomethylketone (PPDK) or a PPDK analog, or a pharmaceutically acceptable salt or ester thereof. An exemplary listing of suitable lysosome modulators may be found in US Patent Publication No. 2016/0136229, which is herein incorporated by reference in its entirety.

In some embodiments, when performing a combination therapy, the two or more drugs can be administered simultaneously or sequentially in any order. In some embodiments, when at least two drugs are administered sequentially, the duration between the two administrations can be about 1 minute, 5 minutes, 10 minutes, 20 minutes, 30 minutes, 1 hour, 2 hours, 4 hours, 6 hours, 12 hours, 24 hours, 2 days, three days, 1 week, 2 weeks, 3 weeks, 1 month, 2 months, 3 months, or more.

In some embodiments, the methods of the present invention further comprise a surgery to be performed on the subject. In some embodiments, the surgery is stem cell transplantation and/or organ transplantation. In some embodiments, the stem cell transplantation is autologous (e.g., stem cells derived from the subject).

In some embodiments, the methods further comprise providing a supportive treatment to the subject. In some embodiments, when the heart or kidneys of the subject are affected, the methods comprise taking a diuretic (water excretion pill), restricting the amount of salt in diet, and/or wearing elastic stockings and elevating their legs to help lessen the amount of swelling. In some embodiments, when the gastrointestinal tract is involved, dietary changes and certain medications can be tried to help symptoms of diarrhea and stomach fullness.

A pharmaceutical composition of the present invention can be administered to a patient by any suitable methods known in the art. In some embodiments, administration of a composition of the present invention may be carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, aerosolly (e.g., inhalation) or by application to mucous membranes.

In some embodiments, a pharmaceutical composition of the present invention further comprises a pharmaceutically-acceptable carrier. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

Compositions intended for oral use may be prepared in either solid or fluid unit dosage forms. Fluid unit dosage form can be prepared according to procedures known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. An elixir is prepared by using a hydroalcoholic (e.g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions contain active materials in admixture with excipients suitable for the manufacture of aqueous suspensions. Such excipients are suspending agents, for example sodium carboxylmethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl-p-hydroxy benzoate, one or more colouring agents, one or more flavoring agents or one or more sweetening agents, such as sucrose or saccharin.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and colouring agents, may also be present.

Pharmaceutical compositions of the invention may also be in the form of oil-in-water emulsions. The oil phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

The pharmaceutical compositions may be in the form of a sterile injectable aqueous or oleaginous suspension. This suspension may be formulated according to known art using those suitable dispersing or wetting agents and suspending agents that have been mentioned above. The sterile injectable preparation may also be a sterile injectable solution or a suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. Adjuvants such as local anaesthetics, preservatives and buffering agents can also be included in the injectable solution or suspension.

In some embodiments, the delivery systems suitable include time-release, delayed release, sustained release, or controlled release delivery systems. In some embodiments, a composition of the present invention can be delivered in a controlled release system, such as sustained-release matrices. Non-limiting examples of sustained-release matrices include polyesters, hydrogels (e.g., poly(2-hydroxyethyl-methacrylate) as described by Langer et al., 1981, J. Biomed. Mater. Res., 15:167-277 and Langer, 1982, Chem. Tech., 12:98-105), or poly(vinylalcohol)], polylactides (U.S. Pat. No. 3,773,919; EP 58,481), copolymers of L-glutamic acid and gamma ethyl-L-glutamate (Sidman et al., 1983, Biopolymers, 22:547-556), non-degradable ethylene-vinyl acetate (Langer et al., supra), degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid (EP 133,988). In some embodiments, the composition may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, for example liver, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990). In some embodiments, the composition may be administered through subcutaneous injection.

In some embodiments, the release of the composition occurs in bursts. Examples of systems in which release occurs in bursts includes, e.g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme.

In some embodiments, the release of the composition is gradual/continuous. Examples of systems in which release of the inhibitor is gradual and continuous include, e.g., erosional systems in which the composition is contained in a form within a matrix and effusional systems in which the composition is released at a controlled rate, e.g., through a polymer. Such sustained release systems can be e.g., in the form of pellets, or capsules.

Other embodiments of the compositions administered according to the invention incorporate particulate forms, protective coatings, protease inhibitors or permeation enhancers for various routes of administration, such as parenteral, pulmonary, nasal and oral. Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philidelphia, Pa. (2000). In some embodiments, the pharmaceutical composition may further include a pharmaceutically acceptable diluent, excipient, carrier, or adjuvant.

In some embodiments, the dosage to be administered is not subject to defined limits, but it will usually be an effective amount, or a therapeutically/pharmaceutically effective amount. The term “effective amount” refers to the amount of one or more compounds that renders a desired treatment outcome. An effective amount may be comprised within one or more doses, i.e., a single dose or multiple doses may be required to achieve the desired treatment endpoint. The term “therapeutically/pharmaceutically effective amount” as used herein, refers to the level or amount of one or more agents needed to treat a condition, or reduce or prevent injury or damage, optionally without causing significant negative or adverse side effects. It will usually be the equivalent, on a molar basis of the pharmacologically active free form produced from a dosage formulation upon the metabolic release of the active free drug to achieve its desired pharmacological and physiological effects. In some embodiments, the compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, dosing regimen of a pharmaceutical composition of the present invention includes, without any limitation, the amount per dose, frequency of dosing, e.g., per day, week, or month, total amount per dosing cycle, dosing interval, dosing variation, pattern or modification per dosing cycle, maximum accumulated dosing, or warm up dosing, or any combination thereof.

In some embodiments, dosing regimen includes a pre-determined or fixed amount per dose in combination with a frequency of such dose. For example, dosing regimen includes a fixed amount per dose in combination with the frequency of such dose being administered to a subject.

In some embodiments, the at least one catabolic enzyme (e.g., PPCA, NEU1, TPP1, cathepsin B, cathepsin D, cathepsin E, cathepsin K, and/or cathepsin L) is administered at about 0.1 to 20 mg/kg daily, weekly, biweekly, monthly, or bi-monthly. In some embodiments, the at least one intralysosomal catabolic enzyme is administered at about 0.2 to 15 mg/kg, about 0.5 to 12 mg/kg, about 1 to 10 mg/kg, about 2 to 8 mg/kg, or about 4 to 6 mg/kg daily, weekly, biweekly, monthly, or bi-monthly.

Based on the suitable dosage, the at least one catabolic enzyme can be provided in various suitable unit dosages. For example, a catabolic enzyme can comprise a unit dosage for administration of one or multiple times per day, for 1-7 days per week, or for 1-31 times per month. Such unit dosages can be provided as a set for daily, weekly and/or monthly administration.

As will be appreciated by those skilled in the art, the duration of the treatment methods depends on the type of amyloidosis being treated, any underlying diseases associated with amyloidosis, the age and conditions of the subject, how the subject responds to the treatment, etc.

In some embodiments, a person having risk of developing amyloidosis (e.g., a person who is genetically predisposed or previously had amyloidosis or associated diseases) can also receive prophylactic treatment of the present invention to inhibit or delay the development of amyloidosis and/or associated diseases.

The pharmaceutical composition of the present invention may also alleviate, reduce the severity of, or reduce the occurrence of, one or more of the symptoms associated with amyloidosis. In some embodiments, the symptoms are those associated with light-chain (AL) amyloidosis (primary systemic amyloidosis) and/or AA amyloidosis (secondary amyloidosis). In some embodiments, the symptoms include, but are not limited to, fluid retention, swelling, shortness of breath, fatigue, irregular heartbeat, numbness of hands and feet, rash, shortness of breath, swallowing difficulties, swollen arms or legs, esophageal reflux, constipation, nausea, abdominal pain, diarrhea, early satiety, stroke, gastrointestinal disorders, enlarged liver, diminished spleen function, diminished function of the adrenal and other endocrine glands, skin color change or growths, lung problems, bleeding and bruising problems, decreased urine output, diarrhea, hoarseness or changing voice, joint pain, and weakness. In some embodiments, the symptoms are those associated with amyloid-beta (Aβ) amyloidosis. In some embodiments, the symptoms include, but are not limited to, common symptoms of Alzheimer's disease, including memory loss, confusion, trouble understanding visual images and spatial relationships, and problems speaking or writing.

In some embodiments, the methods further comprise monitoring the response of the subject after administration to avoid severe and/or fatal immune-mediated adverse reactions due to over-dosage. In some embodiments, the administration of a pharmaceutical composition of the present invention is modified, such as reduced, paused or terminated if the patient shows persistent adverse reactions. In some embodiments, the dosage is modified if the patient fails to respond within about 1 day, 2 days, 3 days, 4 days, 5 days, 6 days, 1 week, 2 weeks or more from administration of first dose.

In some embodiments, a pharmaceutical composition of the present invention can ameliorate, treat, and/or prevent one or more conditions or associated symptoms described herein in a clinically relevant, statistically significant and/or persistent fashion. In some embodiments, administration of a pharmaceutical composition of the present invention provides statistically significant therapeutic effect for ameliorating, treating, and/or preventing one or more symptoms of amyloidosis. In one embodiment, the statistically significant therapeutic effect is determined based on one or more standards or criteria provided by one or more regulatory agencies in the United States, e.g., FDA or other countries. In some embodiments, the statistically significant therapeutic effect is determined based on results obtained from regulatory agency approved clinical trial set up and/or procedure.

In some embodiments, the statistically significant therapeutic effect is determined based on a patient population of at least 50, 100, 200, 300, 400, 500, 600, 700, 800, 900, 1000, or more. In some embodiments, the statistically significant therapeutic effect is determined based on data obtained from randomized and double blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined on approval of Phase III clinical trial of the methods provided by the present invention, e.g., by FDA in the US.

In some embodiment, the statistically significant therapeutic effect is determined by a randomized double blind clinical trial of a patient population of at least 50, 100, 200, 300 or 350; treated with a pharmaceutical composition of the present invention, but not in combination with any other agent. In some embodiment, the statistically significant therapeutic effect is determined by a randomized clinical trial of a patient population of at least 50, 100, 200, 300 or 350 and using any commonly accepted criteria for amyloidosis symptoms assessment.

In general, statistical analysis can include any suitable method permitted by a regulatory agency, e.g., FDA in the US or China or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

The invention also provides packaged pharmaceutical compositions or kits. In some embodiments, the packaged pharmaceutical compositions or kits include a therapeutically effective amount of an intralysosomal catabolic enzyme or a formulation comprising an intralysosomal catabolic enzyme of the present invention described herein. In some embodiments, the compound or formulation can increase the expression, activity, and/or concentration of at least one intralysosomal catabolic enzyme in a subject when the composition is administered to the subject. In some embodiments, the packaged pharmaceutical compositions or kits further comprise in combination with a label or insert advising that the pharmaceutical compound or formulation be administered in combination with a second agent for treating or preventing amyloidosis described herein.

In some embodiments, the packaged pharmaceutical compositions or kits further comprise a therapeutically effective amount of a second agent described herein. In some embodiments, the packaged pharmaceutical compositions or kits is packaged in combination with a label or insert advising that the second agent be administered in combination with the intralysosomal catabolic enzyme or the formulation comprising an intralysosomal catabolic enzyme, or the compound or formulation that can increase the expression, activity, and/or concentration of at least one intralysosomal catabolic enzyme in a subject.

As used herein, the term “label or insert” includes, but is not limited to all written, electronic, or spoken communication with the subject, or with any person substantially responsible for the care of the subject, regarding the administration of the compositions of the present invention. An insert may further include information regarding co-administration of the compositions of the present invention with other compounds or compositions. Additionally, an insert may include instructions regarding administration of the compositions of the present invention before, during, or after a meal, or with/without food.

The following examples illustrate various aspects of the invention. The examples should, of course, be understood to be merely illustrative of only certain embodiments of the invention and not to constitute limitations upon the scope of the invention.

### EXAMPLES

### Example 1: Degradative Effects of Intralysosomal Catabolic Enzymes on Synthetic Amyloid Species

In this example, an in vitro study is performed to illustrate that intralysosomal enzymes such as PPCA (i.e., cathepsin A), cathepsin B, cathepsin D, and/or cocktail mixtures of two or more intralysosomal enzymes can be used for the treatment of amyloidosis. Without being bound by theory, it is hypothesized that delivery of PPCA, cathepsin B, cathepsin D, and other intralysosomal enzymes to lysosomes can assist in the degradation of abnormally accumulated amyloid species, e.g., Aβ-amyloid species before they can be transported into the extracellular space by exocytosis and be deposited as amyloid plaques.

This in vitro study shows the degradative effects of PPCA, cathepsin B, and cathepsin D on synthetic Aβ-amyloid species in a test tube.

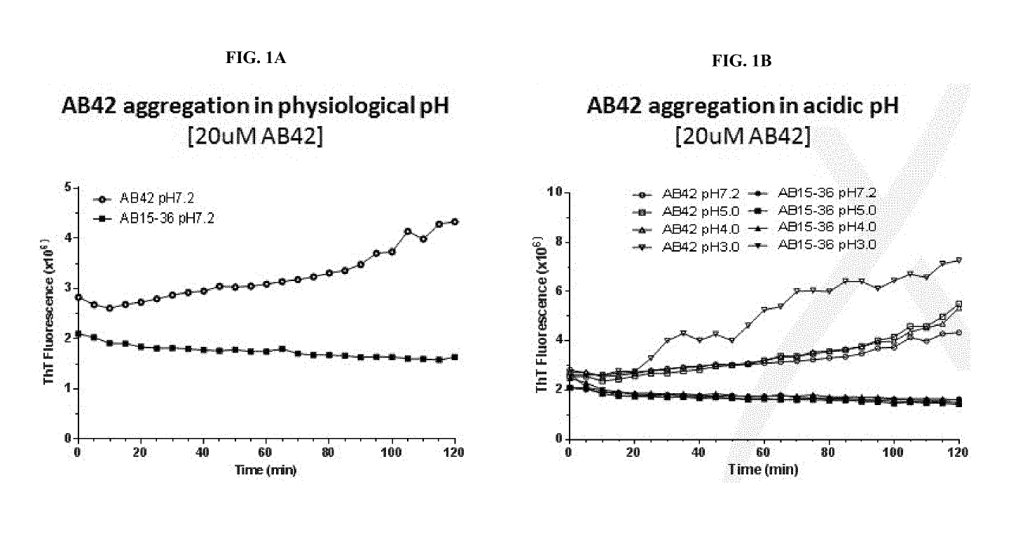

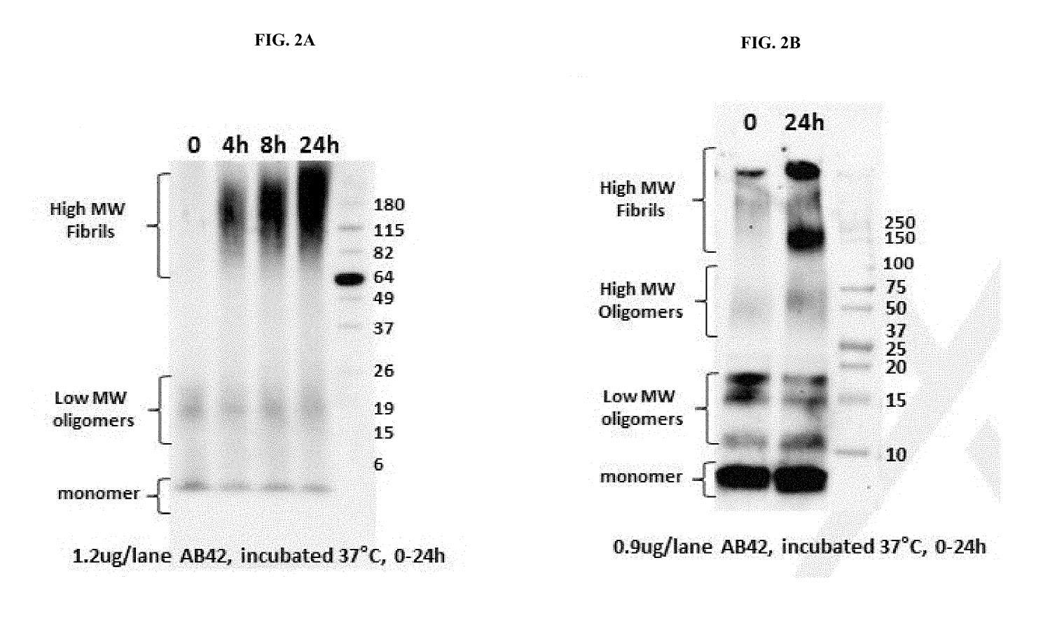

First, in vitro aggregation assays of Aβ-amyloid species using synthetic Aβ-peptides is performed via a Thioflavin-T (THT) assay and western blot. FIG. 1 shows the aggregation of synthetic Aβ42 peptide and Aβ15-36 peptide (negative control) monitored by Thioflavin-T (THT) at physiological conditions (FIG. 1A) or an acidic pH (FIG. 1B). FIG. 2 shows the aggregation of Aβ42 amyloid species over time 24 hours as detected by western blot.

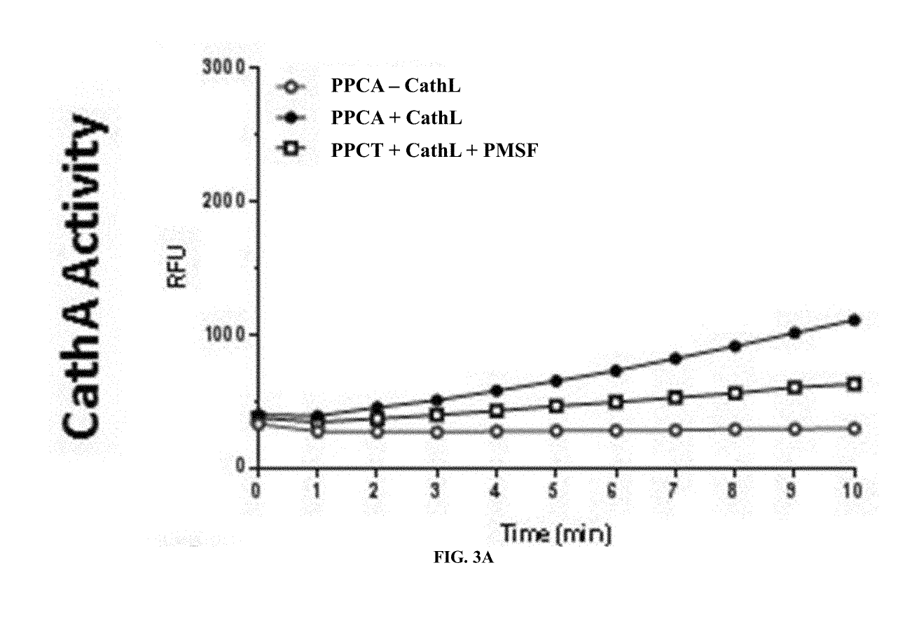

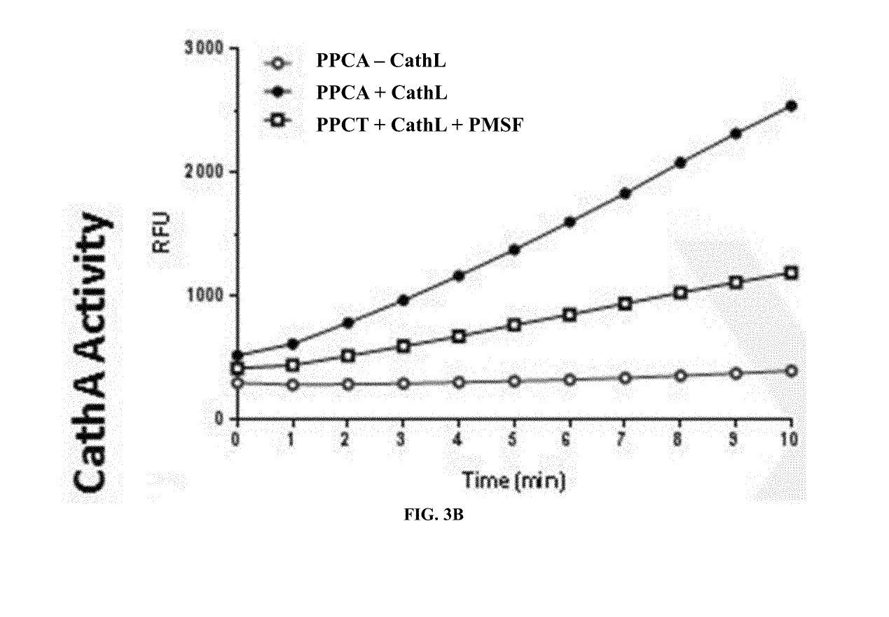

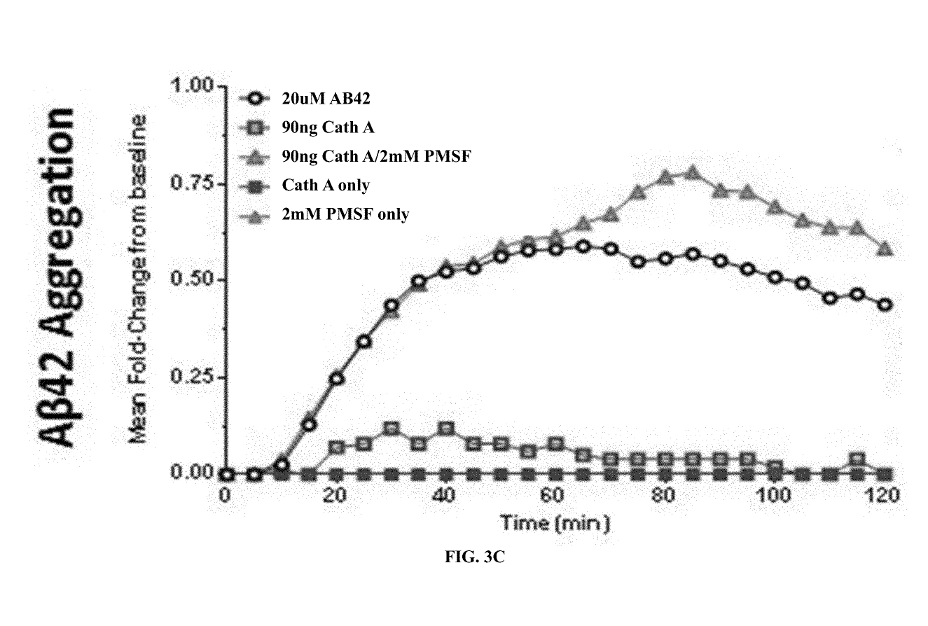

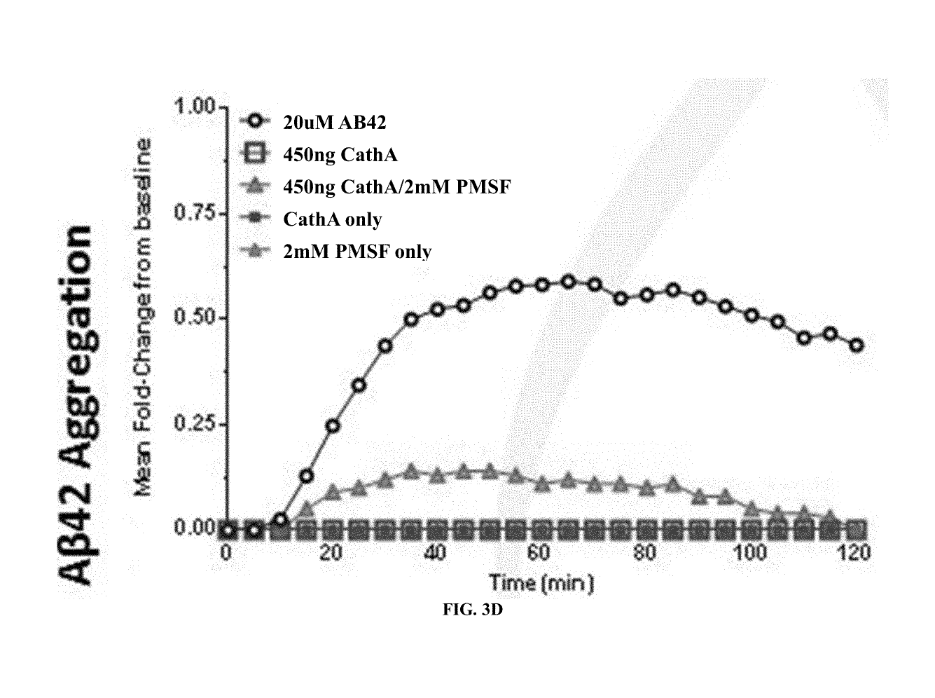

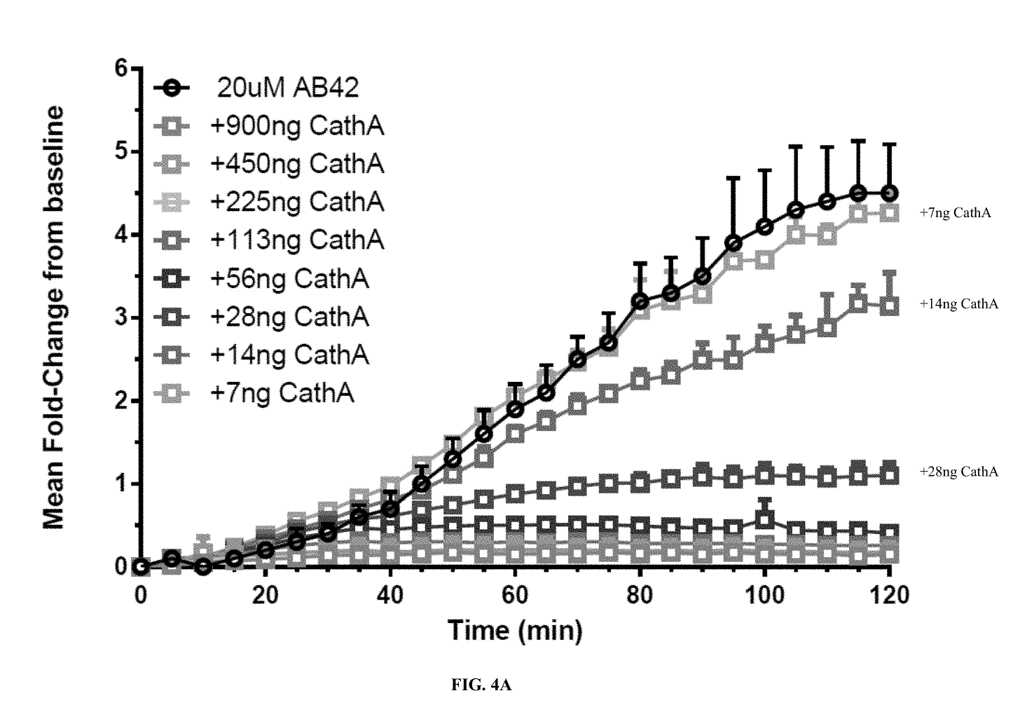

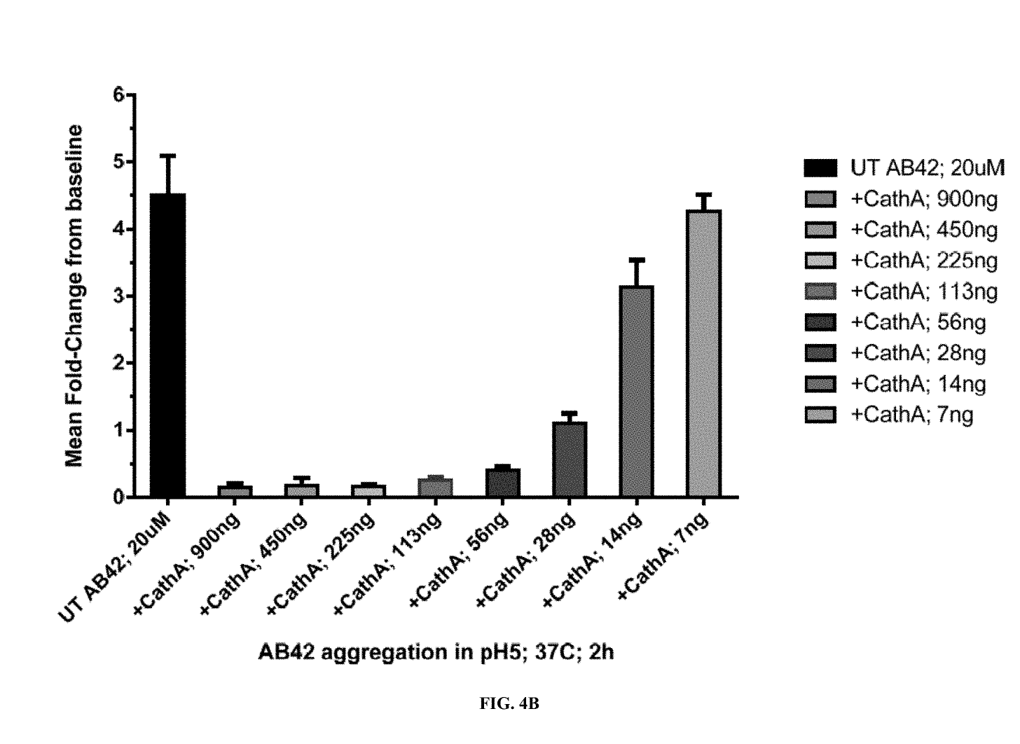

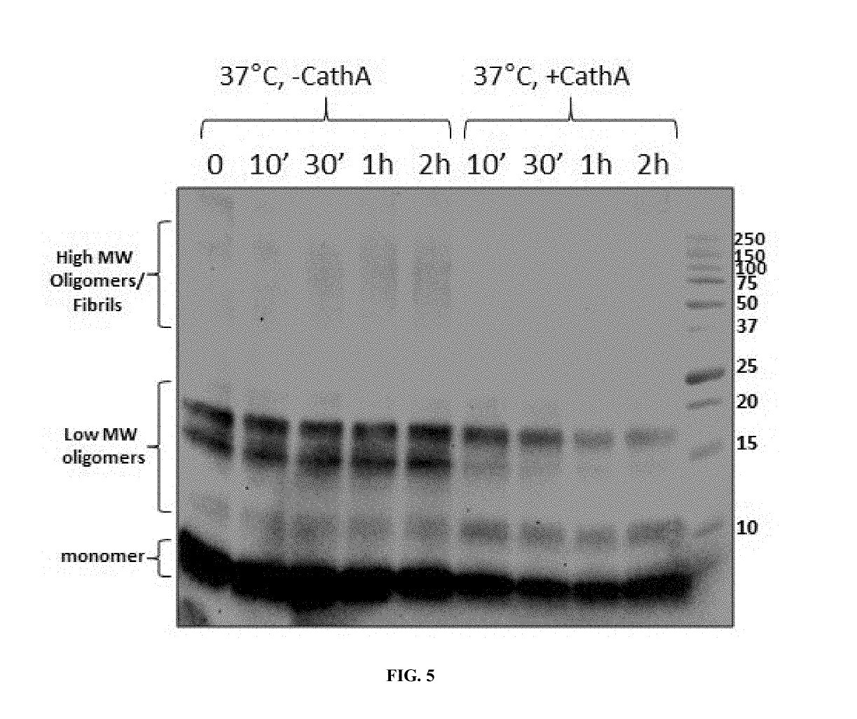

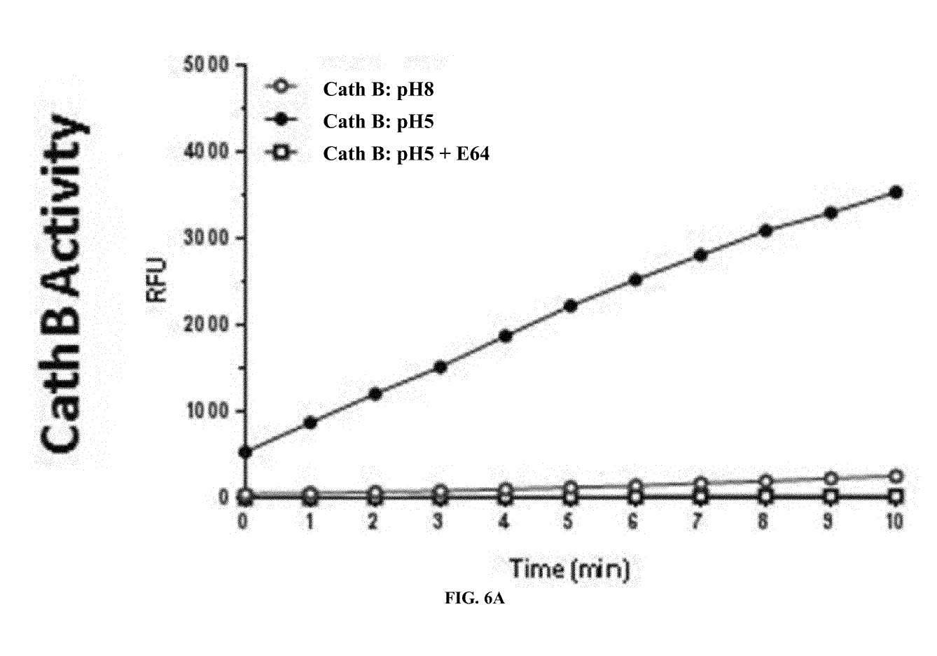

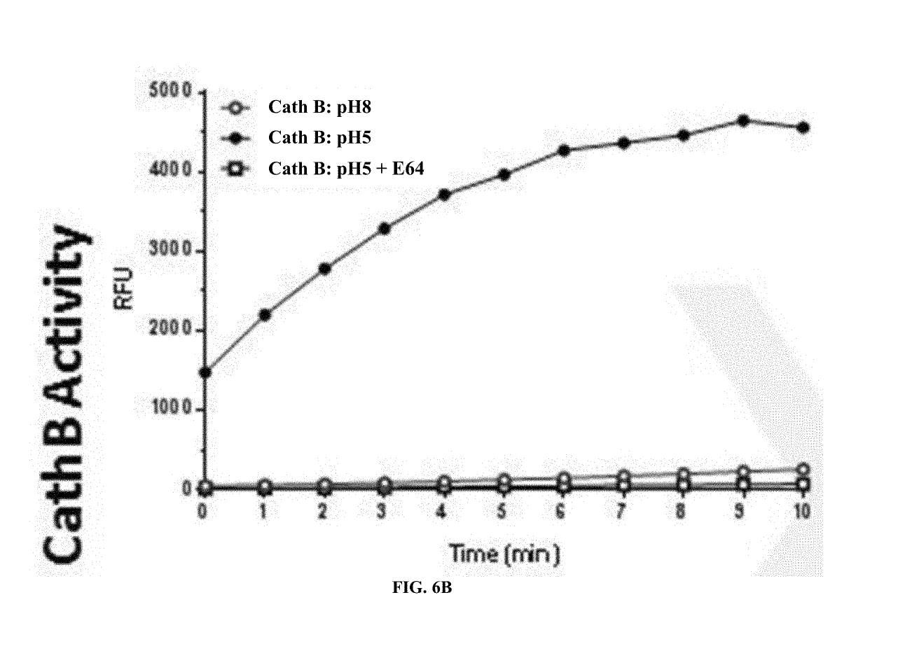

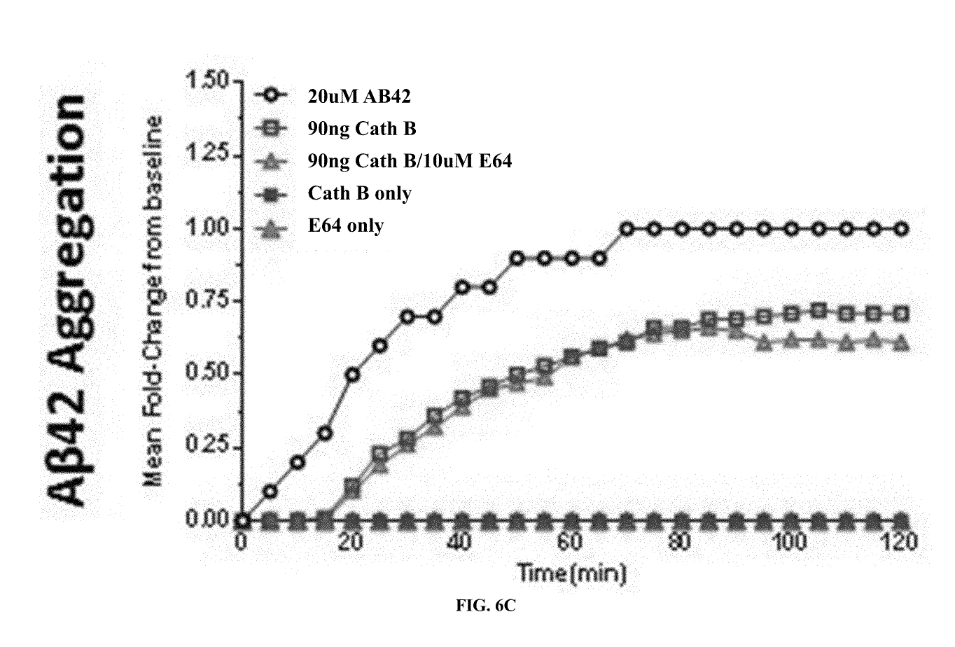

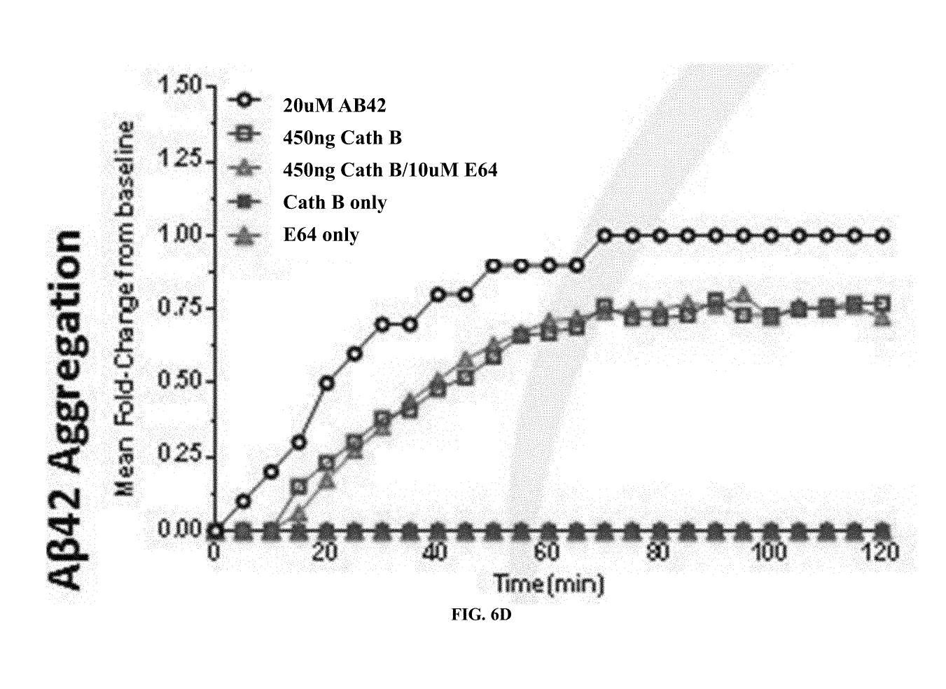

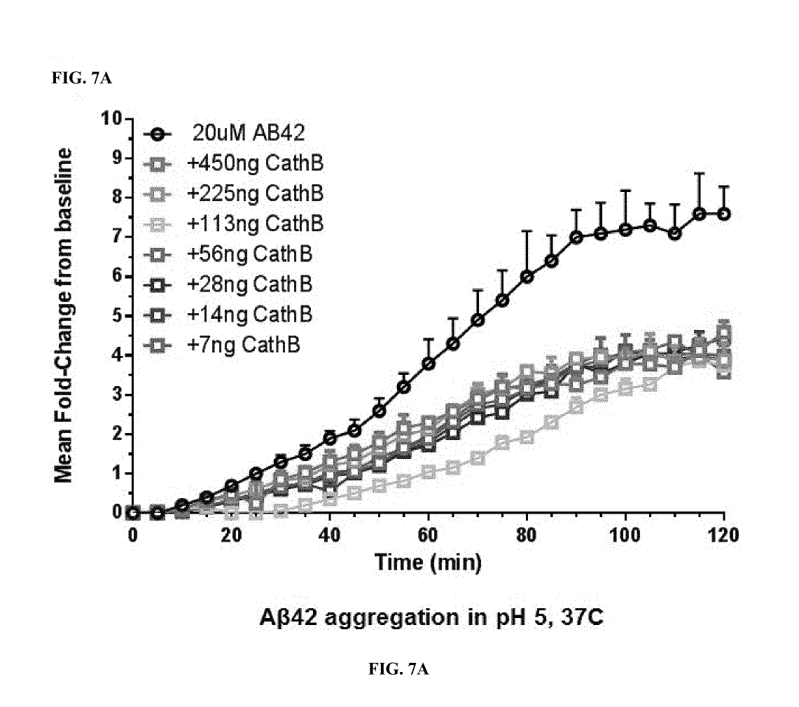

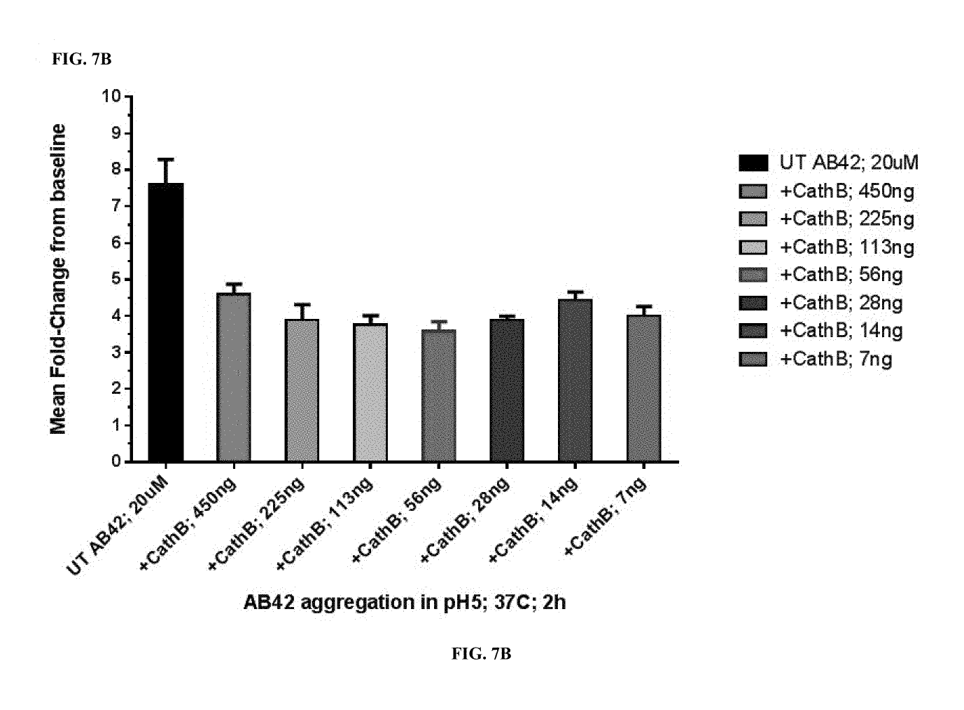

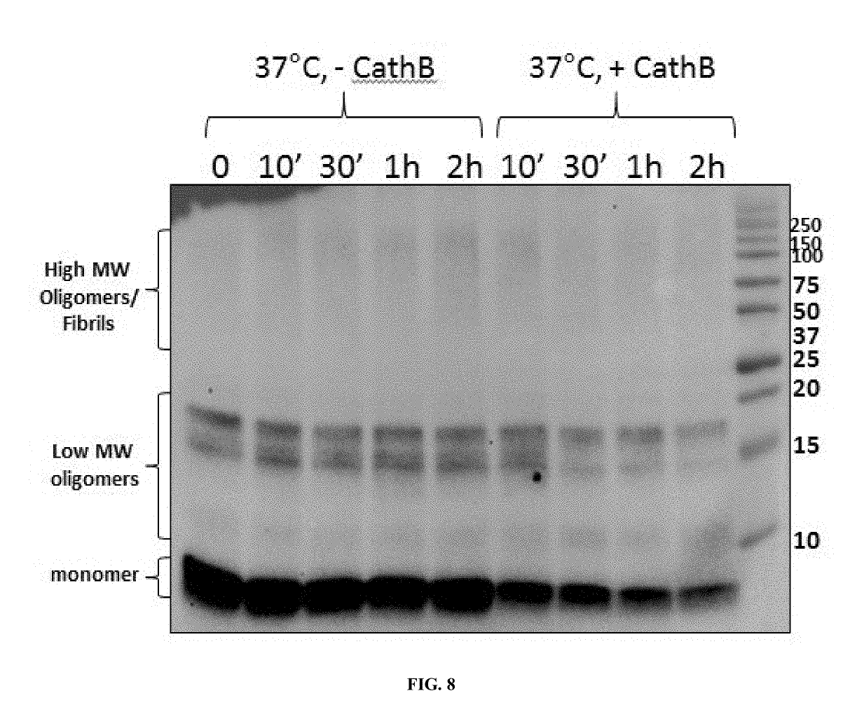

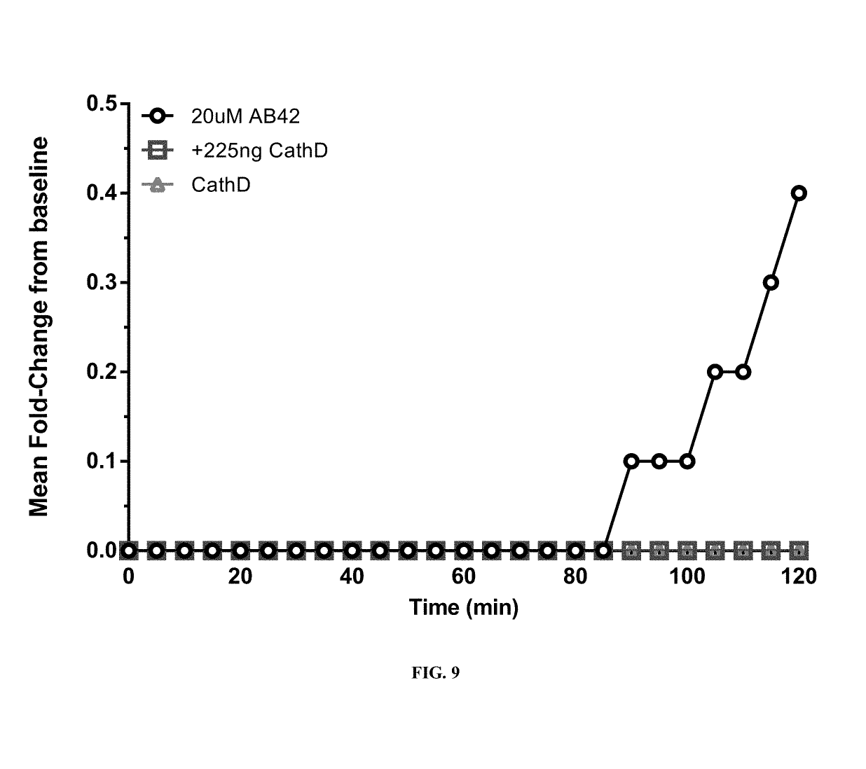

Second, prevention of the aggregation of synthetic Aβ-amyloid species by proteolytic degradation using PPCA, cathepsin B, and cathepsin D is tested via a Thioflavin-T (THT) assay and western blot. FIG. 3 shows that cathepsin A (i.e., PPCA) prevents the aggregation of Aβ42 amyloid. FIG. 4 shows that PPCA prevents the aggregation of Aβ42 amyloid in a dose dependent manner. FIG. 5 shows that PPCA prevents the aggregation of both high and low molecular weight species of Aβ42 amyloid. FIG. 6 shows that cathepsin B prevents the aggregation of Aβ42 amyloid. FIG. 7 shows that cathepsin B moderately prevents the aggregation of Aβ42 amyloid in a dose dependent manner. FIG. 8 shows that cathepsin B prevents the aggregation of low molecular weight species of Aβ42 amyloid and degrades Aβ42 monomers in a time-dependent manner. FIG. 9 shows that cathepsin B prevents the aggregation of Aβ42 amyloid.

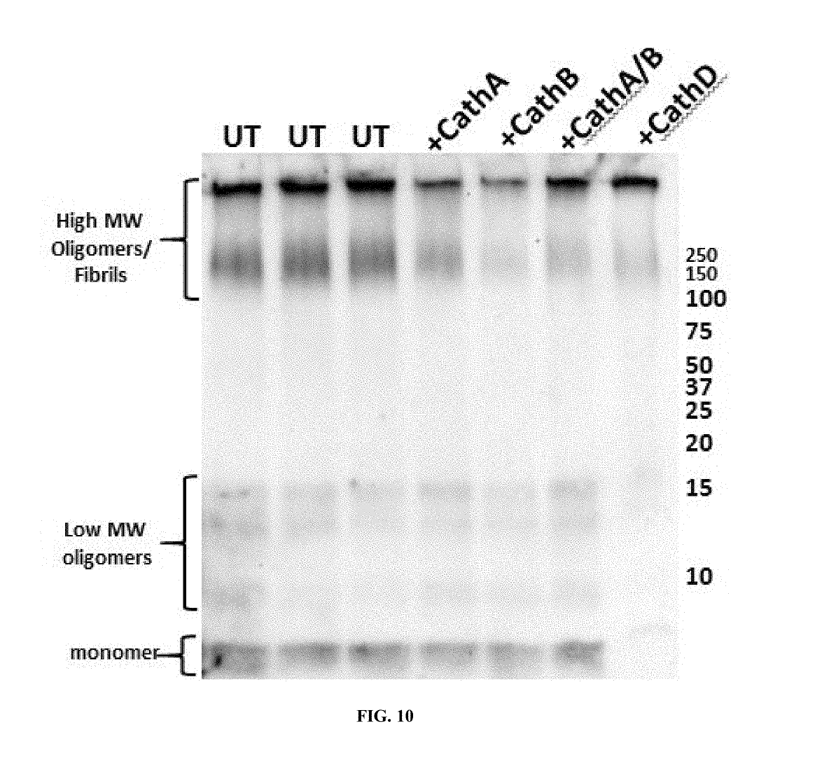

Lastly, the ability of PPCA, cathepsin B, and cathepsin D to degrade pre-formed synthetic A-amyloid species was tested. FIG. 10 shows that PPCA, cathepsin B, PPCA plus cathepsin B, and cathepsin D degrade high molecular weight oligomers/fibrils of Aβ42 amyloid. Cathepsin D degrades low molecular oligomers and completely eliminates Aβ42 monomers.

### Example 1 Summary

Experiments in Example 1 were designed to determine (1) whether the selected intralysosomal catabolic enzymes can prevent aggregation/formation of Aβ amyloid species (called prevention) and (2) whether the selected intralysosomal catabolic enzymes can degrade already pre-formed Aβ amyloid species (called degradation). Example 1 experiments have shown that Aβ42 amyloid species can be aggregated in vitro using synthetic Aβ42 peptides, and that this process can be monitored by THT assay (FIG. 1) and/or western blot analysis (FIG. 2). The THT assay allows for the monitoring of dynamic changes in Aβ42 aggregation upon treatment with degradative enzymes.

Data obtained from the experiments of Example 1 reveal that PPCA can efficiently prevent formation of Aβ42 amyloid species as shown by THT assay (FIG. 3, FIG. 4) and western blot (FIG. 5), as well as degrade already pre-formed amyloid species (FIG. 10). Prevention of amyloid formation and degradation by PPCA was efficient, reproducible and showed concentration dependent dynamics (FIG. 4). Data obtained from experiments with cathepsin B showed moderate reduction in amyloid species formation as measured by THT (FIG. 6). Western blot analysis revealed that cathepsin B prevents aggregation of low molecular weight Aβ42 species and degrades Aβ42 monomers in a time dependent manner (FIG. 8). Experiments with the use of cathepsin D revealed strong prevention of aggregation of Aβ42 species, measured by THT (FIG. 9). Cathepsin D also showed degradation of low molecular oligomers in pre-aggregated amyloid species and complete elimination Aβ42 monomers (FIG. 10).

### Example 2: Degradation of Aβ42 Oligomers and Fibrils by Cathepsin A, B, and D

In this example, two protocols specific for oligomer and fibril formation were applied to aggregate amyloid material to investigate which forms of Aβ42 species can be degraded by cathepsin A (PPCA), cathepsin B and cathepsin D. Aggregated oligomers and fibrils were then subjected to an enzymatic treatment followed by western blot analysis.

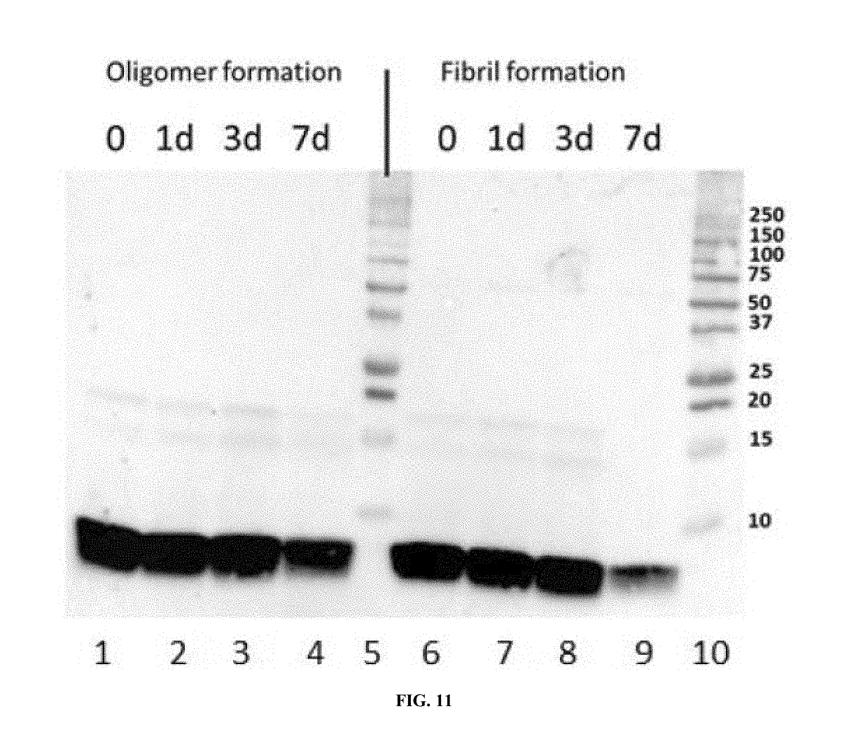

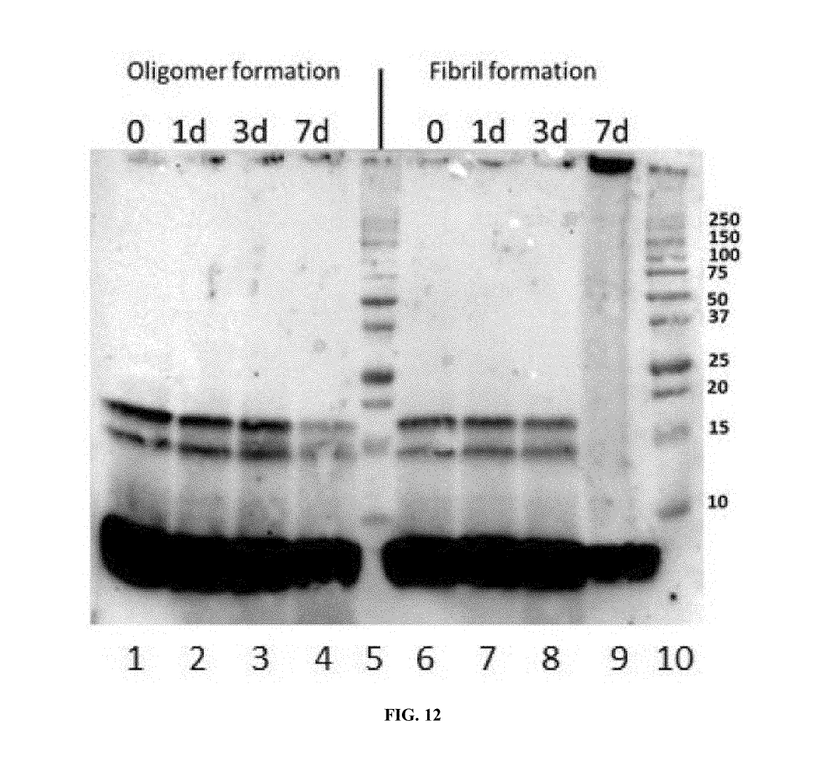

Initially, oligomers and fibrils were aggregated for a period of 7 days and material collected at different time points (days: 0, 1, 3 and 7) was subjected to SDS-PAGE electrophoresis followed by western blot analysis. In FIG. 11, Aβ42 oligomers and Aβ42 fibrils were probed with oligomer specific antibody (A11), which does not recognize monomeric and fibril Aβ42 species. Various forms of oligomers were positively detected on western blot carrying material aggregated using both, oligomer formation and fibril formation protocols. A significant reduction in oligomer forms was observed at day 7 of fibril formation procedure (FIG. 11, line 9), indicating a time dependent transition from oligomers to fibrils, undetectable by A11 antibody. In FIG. 12, the same material as shown in FIG. 11 was probed with E610 antibody, which is specific for both oligomers and fibrils of Aβ42. A lack of fibrils at day 7 was observed when oligomer formation protocol was applied (FIG. 12, line 4) and a strong appearance of fibrils at day 7 when fibril formation protocol was applied.

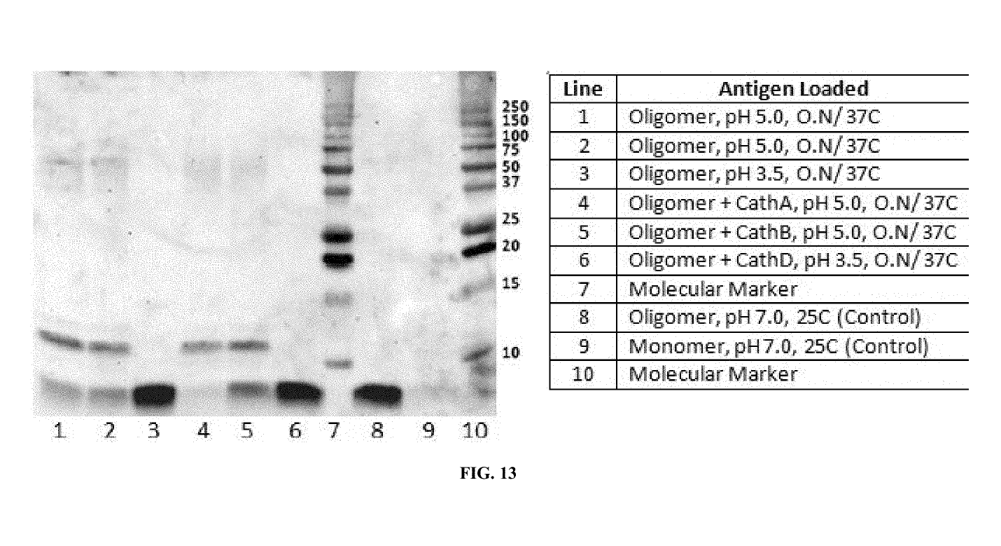

To study enzymatic degradation of oligomer species, Aβ42 oligomers were first aggregated for 9 days at pH 7.0 at 25° C. and then additionally incubated overnight at 37° C. in various pH, optimal for each of enzymes used in the study (pH 5.0 Cathepsin A, B and pH 3.5 Cathepsin D), with and without addition of enzymes. Western blot was probed with oligomer specific A11 antibody (FIG. 13). Additional overnight aggregation of oligomers was observed at pH 5.0 as indicated by presence of higher molecular weight oligomers (lines 1, 2, 4, and 5) when compared to control line 9 (incubation for 9 days at 25° C.). In contrast, this aggregation was not observed for oligomers incubated overnight at pH 3.5. Overnight treatment of oligomers with 90 ng of cathepsin A at pH 5.0 and 37° C. resulted in degradation of the lowest oligomer band (line 4). Treatment of oligomers with 90 ng of cathepsin B and D did not reveal changes in intensity or size of oligomer band (lines 5, 6).

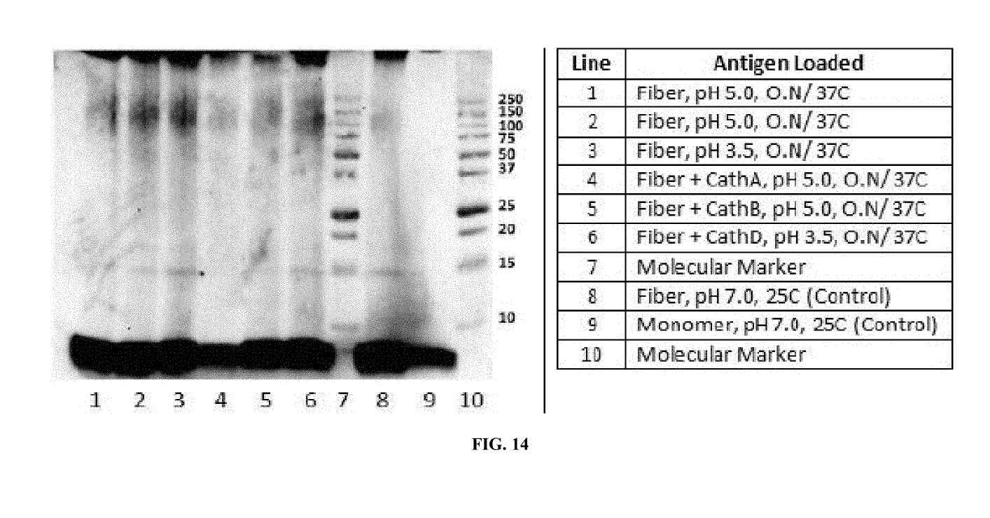

To study enzymatic degradation of fibril species, Aβ42 fibrils were first aggregated for 9 days at pH 7.0 at 25° C. and then additionally incubated overnight at 37 C in various pH, optimal for each of enzymes used in the study (pH 5.0 cathepsin A, B and pH 3.5 cathepsin D), with and without addition of enzymes. Western blot was probed with oligomer specific E610 antibody (FIG. 14). Additional overnight aggregation of fibrils was observed in all pHs applied, as indicated by the presence of stronger/darker smear (lines 1, 2, 3) when compared to control line 9 (incubation for 9 days at 25° C.). Overnight treatment of fibrils with 90 ng of cathepsin A at pH 5.0 and 37° C. resulted in reduction/degradation of the fibril smear as well as degradation of oligomer species (line 4 compared to line 1). Overnight treatment of fibrils with 90 ng of cathepsin B at pH 5.0 and 37° C. resulted in weak reduction/degradation of the fibril smear (line 5 compared to line 2). Overnight treatment of fibrils with 90 ng of cathepsin D at pH 3.5 and 37° C. did not result in visible reduction/degradation of fibril smear or oligomer bands.

### Example 3: Degradation of Aβ42 Monomers by Cathepsin A Monitored by ELISA

The purpose of this example is to assess whether cathepsin A can degrade Aβ42 peptides (monomers).

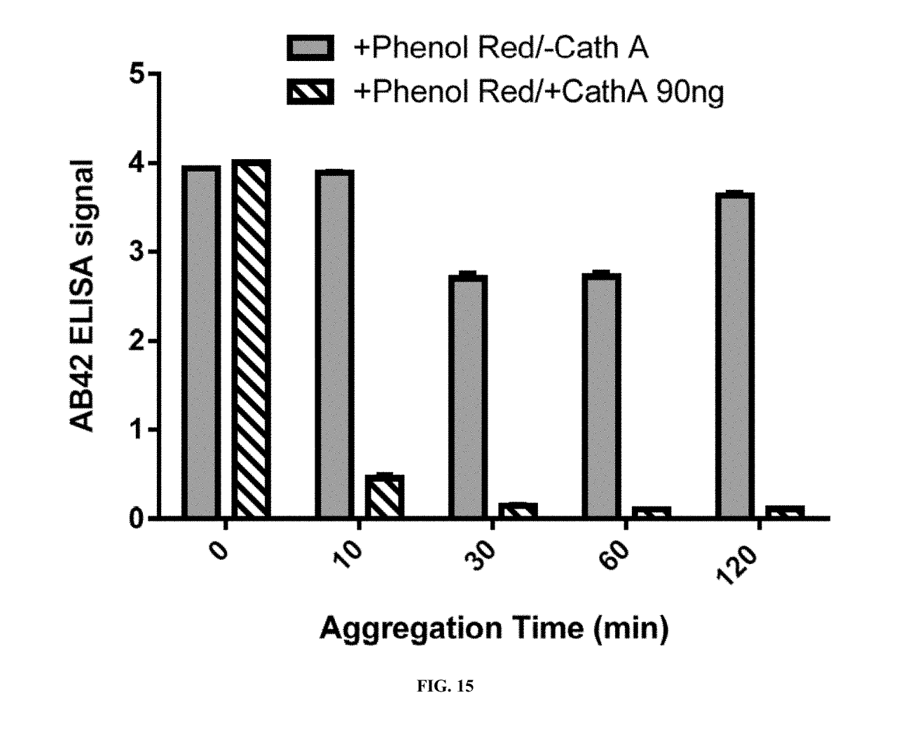

In this example, an enzymatic treatment of peptides with 90 ng of cathepsin A was carried out for 0-2 hr at 37° C. and pH 5.0. An identical experiment without the addition of cathepsin A was performed in parallel. In both cases, phenol red, an inhibitor of Aβ aggregation was used to prevent peptide aggregation into higher molecular weight species of amyloid. The effects of supplementation or lack of cathepsin A on Aβ42 monomers were measured using commercially available ELISA (SensoLyte® Anti-Human β-Amyloid (1-42) Quantitative ELISA, Colorimetric) at various time points (0, 10, 30, 60, 120 min). Sensolite ELISA consists of two antibodies: C-terminal capture antibody, which recognizes specifically human Aβ42 peptide but not Aβ40 or Aβ41 and N-terminal detection antibody. Because Cathepsin A is a carboxyl peptidase, Aβ42 monomers, if degraded, will be degraded from their C-terminus. This degradation would result in a lack of C-terminal amino acid 42 and in consequence lack of capture by C-terminus specific antibody, which should be visualized as a loos of fluorescent signal in ELISA. The ELISA read out for samples treated with cathepsin A revealed a loss of fluorescent signal already within first 10 min of treatment indicating degradation of Aβ42 monomers from the C-terminus by cathepsin A (FIG. 15). Samples without supplementation of cathepsin A showed a strong fluorescent signal in ELISA indicating lack of C-terminal degradation in the absence of enzyme and thus efficient capture of Aβ42 monomers by C-terminus antibody.

### Example 4: Degradation of Aβ40 Amyloid Species by Cath A

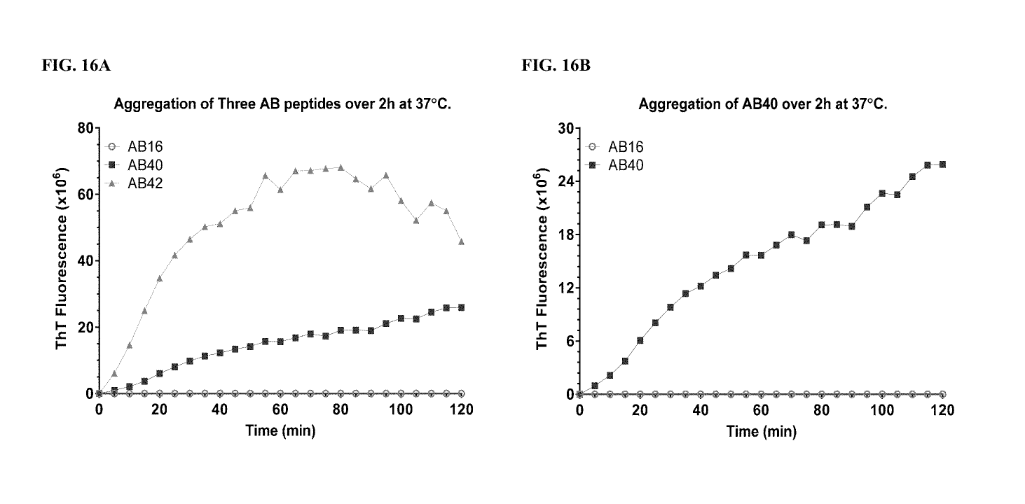

Aggregation experiments showed that Aβ40 amyloid species can be aggregated in vitro using synthetic Aβ40 peptides, and that this process can be monitored by THT assay (FIG. 16). When compared with aggregation of Aβ42 peptides, Aβ40 showed much slower and less efficient rate of aggregation (FIG. 16A).

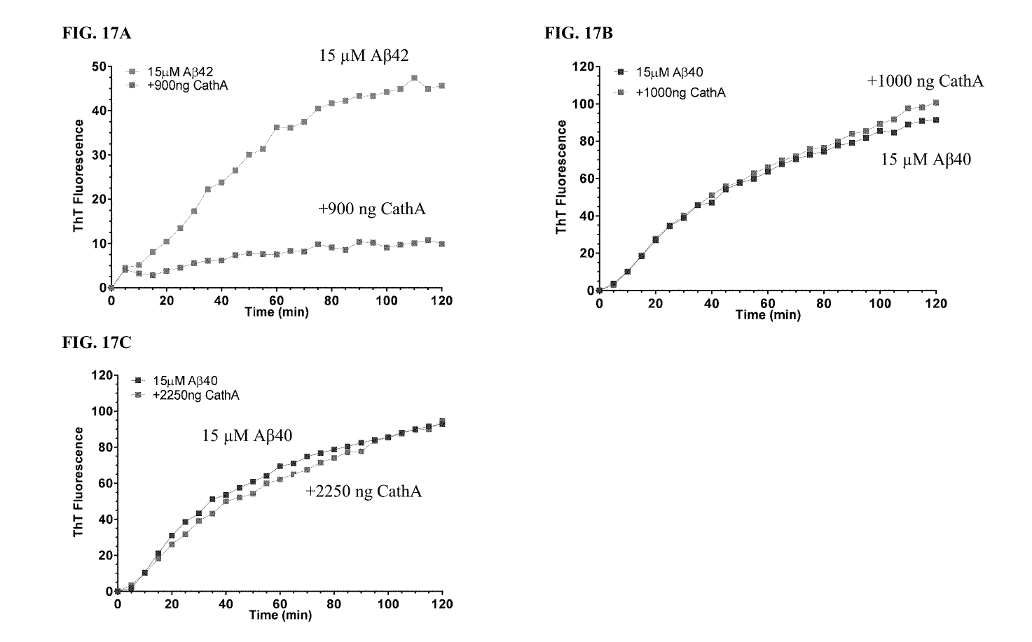

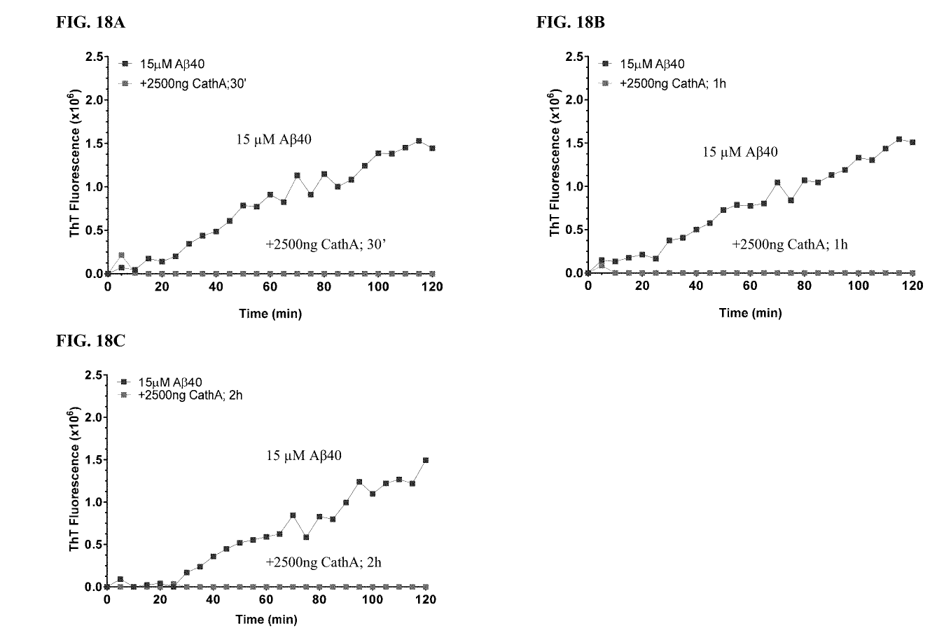

Additional experiments were performed where THT assay was used to monitor dynamic changes in Aβ42 & Aβ40 aggregation upon treatment with degradative enzyme Cath A (FIG. 17). Initial experiment aimed to measure the effect of Cath A treatment on aggregation of both Aβ42 & Aβ40 peptides in real time. To achieve this, Cath A was simultaneously incubated with corresponding peptides and THT reagent in separate reactions at conditions optimal for Cath A proteolysis. The above experiment revealed that in contrast to Aβ42 (FIG. 17A), aggregation of Aβ40 amyloid is not affected by Cath A, in applied experimental settings, even when high concentration of enzyme is used (FIG. 17B, C). Second experiment was carried out to investigate whether the result of the initial experiment is due to lack of proteolysis of Aβ40 by Cath A or whether the speed of such proteolysis is slower than the speed of Aβ40 aggregation and therefore no changes in THT fluorescence could be observed. In this experiment Aβ40 peptide was first incubated with Cath A for up to two hours in conditions optimal for Cath A proteolysis and followed by incubation with THT to measure aggregation. Obtained data revealed that Aβ40 peptide did not aggregate after pre-incubation with Cath A, proving its proteolysis (FIG. 18).

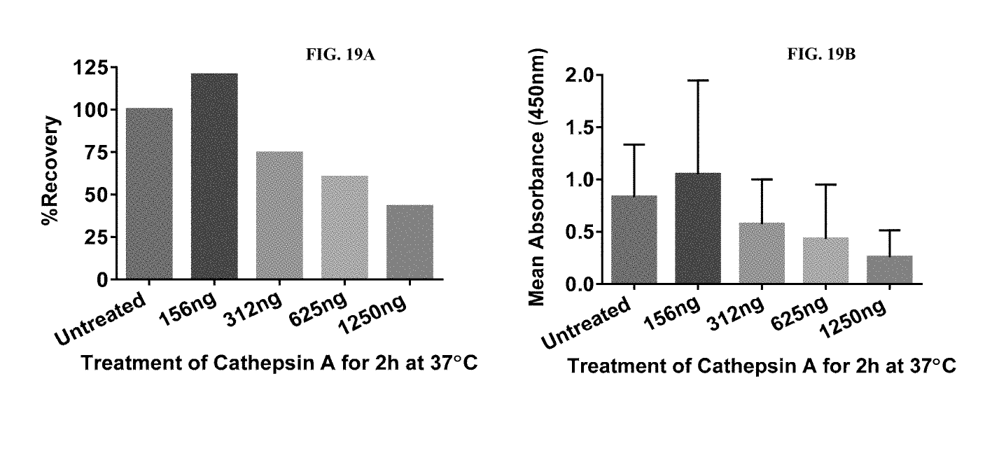

To prove that observed loss of aggregation by Aβ40 peptide is caused by carboxypeptidase activity of Cath A, Aβ40 peptide was incubated for two hours at 37° C. at pH5 with varying concentrations of Cath A. Subsequently, the reaction was transferred to an ELISA plate pre-coated with a C-terminal capture antibody, specifically for Aβ40 peptide only and was co-incubated with N-terminal detection antibody overnight at 4°. The results have shown progressively reduced binding of Aβ40 peptide to C-terminal capture antibody with increasing concentration of Cath A (FIG. 19). This proves that C-terminus of Aβ40 peptide was removed by caboxyterminal activity of Cath A.

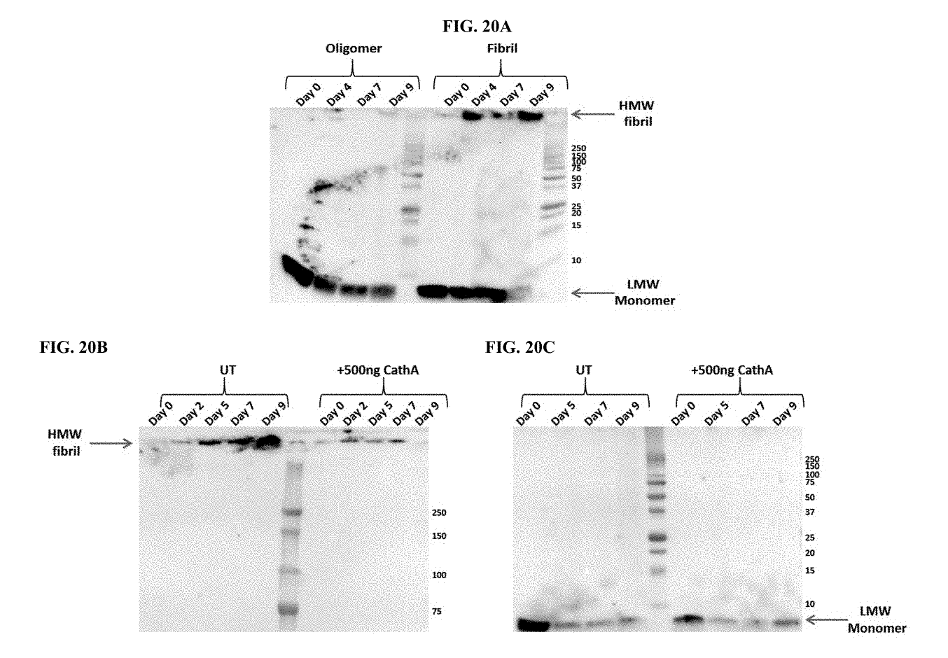

Aggregation of Aβ40 peptide into amyloid species was also monitored using Western Blot technique (FIG. 20A). We were able to aggregate Aβ40 into high molecular weight fibrils but not oligomeric forms using aggregation process taking up to 9 days. An experiment was carried out in which Aβ40 was simultaneously incubated Cath A for up to 9 days during the process of fibril formation. Obtained results revealed that Cath A significantly prevents formation of high molecular weight fibrils due to its proteolytic action on Aβ40 amyloid (FIG. 20B). Reduction of levels of monomeric Aβ40 form was also observed in this experiment (FIG. 20C).

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and the application is intended to cover any variations, uses, or adaptations of the invention following, in general, the principles of the invention and including such departures from the disclosure as come within known or customary practice within the art to which the invention pertains and as may be applied to the essential features set forth and as follows in the scope of the appended claims.

1. A method of treating or preventing AA amyloidosis in a subject comprising administering to the subject a composition comprising a therapeutically effective amount of cathepsin L or a biologically active fragment thereof.

2.-17. (canceled)

18. The method of claim 1, wherein cathepsin L acts to prevent the formation of and/or degrade amyloid within a lysosome.

19. The method of claim 1, wherein cathepsin L is targeted to a cell lysosome.

20.-24. (canceled)

25. The method of claim 1, wherein the subject is a human.

26-48. (canceled)

49. The method of claim 1, wherein the cathepsin L comprises an amino acid sequence with at least 85% sequence identity to SEQ ID NO: 12 or SEQ ID NO: 65.

50. The method of claim 49, wherein the cathepsin L comprises an amino acid sequence selected from SEQ ID NO: 12 and SEQ ID NO: 65.

51. The method of claim 50, wherein the cathepsin L comprises SEQ ID NO: 12.

52. The method of claim 1, wherein the cathepsin L is a recombinant cathepsin L.

53. The method of claim 1, wherein the cathepsin L is administered intravenously.

54. The method of claim 1, wherein the cathepsin L is administered subcutaneously.

55. The method of claim 1, wherein the cathepsin L is administered at a dose of about 0.5 mg/kg to 12 mg/kg.

56. The method of claim 55, wherein the cathepsin L is administered at a dose of about 1 mg/kg to about 10 mg/kg.

57. The method of claim 55, wherein the cathepsin L is administered at a dose of about 2 mg/kg to 8 mg/kg.

58. The method of claim 55, wherein the cathepsin L is administered at a dose of about 4 mg/kg to 6 mg/kg.

59. The method of claim 1, wherein the cathepsin L is administered at a frequency selected from daily, weekly, biweekly, monthly, or bi-monthly.

60. The method of claim 59, wherein the cathepsin L is administered daily.

61. The method of claim 59, wherein the cathepsin L is administered weekly.

62. The method of claim 59, wherein the cathepsin L is administered biweekly.

63. The method of claim 59, wherein the cathepsin L is administered monthly.

64. The method of claim 1, wherein the AA amyloidosis involves one or more organs selected from liver, spleen, and kidney.
